(19) United States
(12) Patent Application Publication
(10) Pub. No.: US 2022/0403451 A1
(43) Pub. Date: Dec. 22, 2022

(54) ON-CHIP ASSAY STRATEGY FOR THE DEVELOPMENT OF ELECTROCHEMICAL READOUT FOR CRISPR-CAS DIAGNOSTICS

(71) Applicants: PRESIDENT AND FELLOWS OF HARVARD COLLEGE, Cambridge, MA (US); MASSACHUSETTS INSTITUTE OF TECHNOLOGY, Cambridge, MA (US)

(72) Inventors: Helena DE PUIG GUIXE, Barcelona (ES); Pawan JOLLY, Boston, MA (US); James J. COLLINS, Newton, MA (US); Donald E. INGBER, Boston, MA (US)

(73) Assignees: PRESIDENT AND FELLOWS OF HARVARD COLLEGE, Cambridge, MA (US); MASSACHUSETTS INSTITUTE OF TECHNOLOGY, Cambridge, MA (US)

(21) Appl. No.: 17/772,782

(22) PCT Filed: Oct. 30, 2020
(86) PCT No.: PCT/US2020/058116

§ 371 Date: Apr. 28, 2022

Related U.S. Application Data
(60) Provisional application No. 62/928,841, filed on Oct. 31, 2019.

(51) Int. Cl. C12Q 1/6825; G01N 27/327; C12N 15/10

(52) U.S. Cl. CPC C12Q 1/6825; G01N 27/327; C12N 15/102; C12N 2310/20

(57) ABSTRACT

This disclosure is generally directed to electrochemical readout of rapid diagnostics related to use of CRISPR effector systems. In one aspect, the disclosure provides a nucleic acid detection system. Generally, the system comprises: (1) a detection CRISPR system comprising an effector protein and one or more guide nucleic acid (gNA) strands designed to bind to corresponding target nucleic acid molecules; (2) an effector strand and (3) an electrode.

### CROSS-REFERENCE TO RELATED APPLICATIONS

This application claims benefit under 35 U.S.C. § 119(e) of U.S. Provisional Application No. 62/928,841 filed Oct. 31, 2019, the content of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 28, 2020 is named 002806-096460WOPT_SL.txt and is 3,525 bytes in size.

### FIELD OF THE INVENTION

This disclosure is generally directed to rapid diagnostics related to use of CRISPR effector systems.

### BACKGROUND

Specific High-Sensitivity Enzymatic Reporter UnLOCKing (SHERLOCK) diagnostic tools (Gootenberg J S, Abudayyeh O O, Lee J W, Essletzbichler P, Dy A J, Joung J, et al. Nucleic acid detection with CRISPR-Cas13a/C2c2. Science. 2017; 356(6336):438-442), have been previously reporter to create highly sensitive and specific CRISPR-based diagnostics. CRISPR-based diagnostics leverage the programmable endonucleases (Cas enzymes) of CRISPR-associated microbial adaptive immune systems. Several Cas enzymes have been used for diagnostic purposes: e.g. Cas13a, Cas14, Cas9, Cas12a. In particular, Cas12a (also known as cpf1) is an RNA-guided, DNA targeting enzyme, which can be reprogrammed with CRISPR guide RNAs (gRNA) to construct modular and highly specific DNA sensing platforms (Chen J S, Ma M, Harrington L, Da Costa M, Tian X, Palefsky J M, Doudna J A, CRISPR-Cas12a target binding unleashes indiscriminate single-stranded DNase activity. Science. 2018).

Cas12a activates through recognition of its dsDNA target. Once activated, it exhibits promiscuous, non-specific DNase activity and cleaves non-target DNAs. Non-target, sacrificial ssDNA contain a fluorophore on the 5′ end and a quencher on the 3′ end. Once Cas12a activates, it collaterally cleaves the non-target ssDNA, thus releasing the fluorophore and leading to increased fluorescence. Typically, this enzymatic, non-specific degradation of non-target labeled ssDNA is used to detect the presence of the dsDNA target that activated the enzyme (see e.g., FIG. 1). Isothermal amplification such as NASBA, RCA, RPA, LAMP or RT-RPA can be coupled with Cas12a detection. Isothermal pre-amplification of target nucleic acids allows one to lower the limit of detection by amplifying the target sequences. Cas12a gives the capacity to detect both genomic DNA, as well as RT-RPA products resulting from RNA amplification.

The readouts of CRISPR/Cas diagnostics, such as SHERLOCK, DETECTR, HUDSON or HOLMES have been typically limited to fluorescence. Fluorescence readouts have several limitations in diagnostic devices: (1) they require an external machine reader, which is typically not portable; (2) they require post-processing of results for machine-interface; and (3) they require trained users and specialized facilities and equipment.

The present disclosure addresses some of these limitations.

### SUMMARY

The disclosure is based on inventors' discovery of a novel method to obtain an electrochemical readout for the CRISPR/Cas diagnostics. Electrochemistry is a highly sensitive and quantitative technique to measure interactions taking place at or near the electrode interface. It is desirable as a readout for diagnostics as it can be made rapid, sensitive, low cost, portable, results can be readily interfaced with machine, and it does not require trained user nor specialized facilities.

In one aspect, the disclosure provides a nucleic acid detection system. Generally, the system comprises: (1) a detection CRISPR system comprising an effector protein and one or more guide nucleic acid (gNA) strands designed to bind to corresponding target nucleic acid molecules; (2) an effector strand and (3) an electrode. Optionally, the effector stand is immobilized on a surface, e.g. a conductive surface of the electrode. Optionally the effector nucleic acid strand is conjugated with at least one electroactive label.

In various embodiments, the electrode comprises a nanocomposite coating comprising a mixture of a conducting element and a denatured proteinaceous material coated on at least a part of a conductive surface of the electrode. In some embodiments, the nanocomposite coating comprises a three dimensional, porous matrix.

In some embodiments, the detection system further comprises a detector nucleic acid strand, wherein the detector nucleic acid stand is substantially complementary to the effector nucleic acid strand and is optionally conjugated with at least one electroactive label.

In another aspect, the disclosure provides a method for detecting a target nucleic acid in sample. Generally, the method comprising contacting a sample suspected of comprising the target nucleic acid with a detection CRISPR system, wherein the detection CRISPR system comprises an effector protein and one or more guide nucleic acid (gNA) strands designed to bind to corresponding target nucleic acid.

If a target molecule is present in a sample, the corresponding guide nucleic acid strand guides the CRISPR Cas/guide complex to the target molecule by hybridizing with the target molecule, thereby triggering the CRISPR effector protein's nuclease activity. This activated CRISPR effector protein cleaves both the target molecule and then non-specifically cleave the effector nucleic acid present on the electrode. The cleavage of the effector nucleic acid stand can be electrochemically detected.

For example, the effector molecule can comprise an electroactive label. The cleavage of the effector nucleic acid stand reduces the number of electroactive labels attached to the electrode. This changes the redox potential of the electrode and this change can be measured electrochemically.

Alternatively, a detector nucleic acid strand having substantial complementarity to the effector nucleic acid strand and comprising at least one electroactive label can be used. While the detect nucleic acid strand can bind to the full length effector molecule, the detector and effectors strands do not bind to each other once the effector strand is cleaved. This reduces the number of electroactive labels bound to the electrode; thereby changing the redox potential of the electrode. This change in the redox potential can be electrochemically measured.

In some embodiments, the activated CRISPR effector protein cleaves the target molecule and then non-specifically cleaves the detector nucleic acid strand. The cleavage of the detector nucleic acid stand can be electrochemically detected by hybridizing any remaining full length detector strands with the effector strand on the electrode.

### BRIEF DESCRIPTION OF THE DRAWINGS

FIG. 1 is a schematic representation of prior art SHERLOCK using Cas12a.

FIG. 2A is a bar graph showing reporter fluorescence measurement after 60-minute reactions at 37° C. Five different gRNA-based Lyme sensors were tested using a 350 pM starting concentration of trigger DNA, or no trigger. Error bars represent SD from three replicates.

FIG. 2B is a bar graph showing reporter fluorescence resulting from increasing concentrations of trigger genomic DNA starting material that was subjected to 40 min RPA at 37° C. followed by a 60-minute Cas12a detection reaction at 37° C., using Cas12a sensors a-e. Error bars represent SD from three replicates.

FIG. 3 is a schematic representation of surface functionalization of an electrode according to an embodiment of the invention.

FIG. 4 is a schematic representation of electrochemical readout of the CRISPR/Cas diagnostics according an embodiment of the invention.

FIGS. 5A and 5B show Cyclic Voltammetry results using electrochemical readouts coupled to SHERLOCK.

FIGS. 6A and 6B show Square Wave Voltammetry results using electrochemical readouts coupled to SHERLOCK.

FIG. 7 is a schematic representation of electrochemical readout of the CRISPR/Cas diagnostics according an embodiment of the invention.

FIG. 8 shows Cyclic Voltammetry results using electrochemical readouts coupled to SHERLOCK to detect SARS-CoV-2 RNA.

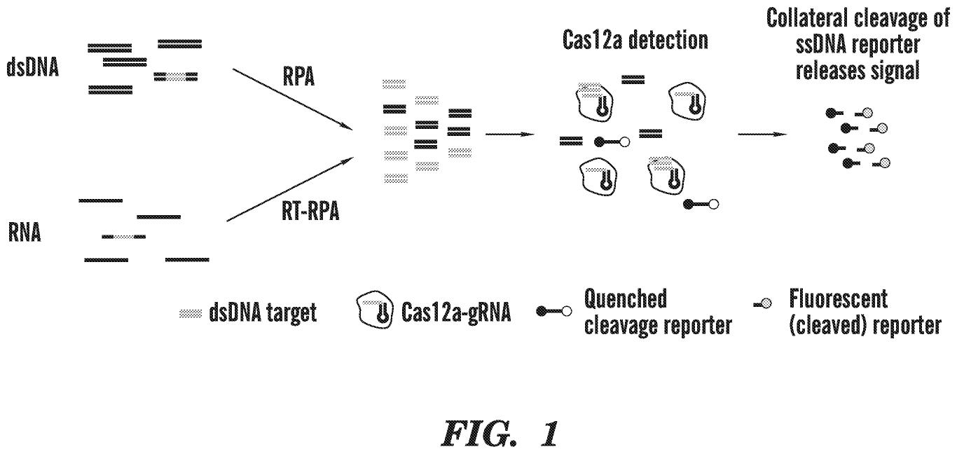

### DETAILED DESCRIPTION

CRISPR-based diagnostic readouts have been typically limited to fluorescence. Inventors have now discovered inter alia a novel method to obtain electrochemical readouts from CRISPR/Cas-based diagnostics. Electrochemical methods are methods that rely on a change in the potential, charge or current to characterize chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

The embodiments disclosed herein utilize RNA or DNA targeting effectors to provide a robust CRISPR-based diagnostic with attomolar sensitivity. Embodiments disclosed herein can detect both DNA and RNA with comparable levels of sensitivity and can differentiate targets from non-targets based on single base pair differences.

In one aspect, the disclosure provides a nucleic acid detection system. Generally, the system comprises: (1) a detection CRISPR system comprising an effector protein and one or more guide nucleic acid (gNA) strands designed to bind to corresponding target nucleic acid molecules; (2) an effector strand; and (3) an electrode. In some embodiments, the nucleic acid detection system further comprises a detector nucleic acid strand, wherein the detector nucleic acid stand is substantially complementary to the effector nucleic acid strand, optionally, the detector nucleic acid strand is conjugated with at least one electroactive label.

### Electrode

As used herein, an “electrode” is an electrical conductor used to make contact with a nonmetallic part of a circuit (i.e., it emits or collects electrons or electron “holes”). Electrodes can comprise any electrically conducting or semi-conducting material. Non-limiting examples include gold, silver, copper, platinum, aluminum, stainless steel, tungsten, indium tin oxide, titanium, lead, nickel, silicon, polyimide, parylene, benzocyclobutene, carbon, graphite, or any combination thereof. Preferably, electrodes comprise gold. The use of inexpensive gold-coated printed circuit board (PCB) substrates as electrochemical sensor platform has been reported [La Belle, J. T., et al., Label-Free Impedimetric Detection of Glycan-Lectin Interactions. Analytical Chemistry, 2007. 79(18): p. 6959-6964. Umek, R. M., et al., Electronic Detection of Nucleic Acids: A Versatile Platform for Molecular Diagnostics. The Journal of Molecular Diagnostics, 2001. 3(2): p. 74-84. Lian, K., et al., Integrated microfluidic components on a printed wiring board platform. Sensors and Actuators B: Chemical, 2009. 138(1): p. 21-27]. Metal patterning techniques, such as standard PCB technology, offer a number of versatile fabrication options such as (i) track size and spacing less than 100 μm; (ii) high purity electrolytic gold plating several microns thick suitable for electrochemistry and surface modification chemistries; (iii) ease of small scale prototyping in standard laboratory settings; and (iv) large scale mass manufacturing capabilities at a fraction of the cost of high-end microarrays. In some embodiments, electrodes as disclosed herein can be fabricated using PCB technology.

In some embodiments, the electrodes are mass fabricated onto non-electrically conductive surfaces such as plastic substrates using inexpensive standard technology such as printed circuit board (PCB) technology, roll-to-roll laser ablation or evaporation. Exemplary non-electrically conductive surfaces include plastic, poly(carbonate) (PC), poly(methyl methacrylate) (PMMA), cyclic olefin polymers (COP) or cyclic olefin copolymers (COC), SU-8, parylene, silicon nitride, kapton, styrene-ethylene-butylene-styrene (SEBS), poly-dimethylsiloxane (PDMS), polyimide, silicon dioxide, and any combination thereof.

In some embodiments, the electrode is a planar or a 3-dimensional electrode. As used herein, a planar electrode electrically interacts with an electroactive species or mediator on a 2-dimensional surface. As used herein, a 3-dimensional electrode is an electrode displaying a very high surface area per unit volume, caused by no planarity. Without being bound by theory, this provides high turbulence at their interface with an electroactive species or mediator, enhancing the mass transfer process of the electroactive species towards the electrode surface. These characteristics strongly improve the electrochemical reaction rate.

The electrode can be large (e.g., with a working surface area of greater than 1 cm², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium.

Types of electrodes include detector electrodes, positive control electrodes, negative control electrodes, counter electrodes, reference electrodes, among other types. As used herein, “detector electrodes” are electrodes coated or otherwise functionalized with effector nucleic acid strands.

Nonspecific binding of effector proteins, e.g., Cas proteins on the electrodes could mask the electrochemical signal. Accordingly, in some embodiments, the surface of the electrode can be coated to reduce or inhibit nonspecific binding of effector proteins, e.g., Cas proteins on the electrodes. Without limitations, such a coating can also allow for high concentration of effector nucleic acid strands to be attached to the electrode. Any coating known in the art for reducing or inhibiting non-specific binding of proteins to a surface can be used.

For example, the surface of the electrode can be coated with a blocking agent. As used herein a “blocking agent” is a compound used to prevent non-specific interactions. The blocking agent can be a protein, mixture of proteins, fragments of proteins, peptides or other compounds that can passively absorb to the surface in need of blocking. For example, proteins (e.g., BSA and Casein), poloxamers (e.g., pluronics), PEG-based polymers and oligomers (e.g., diethylene glycol dimethyl ether), cationic surfactants (e.g., DOTAP, DOPE, DOTMA). Some other examples include commercially available blocking agent or components therein that are available from, for example, Rockland Inc. (Limeric, Pa.) such as: BBS Fish Gel Concentrate; PBS Fish Gel Concentrate; TBS Fish Gel Concentrate; Blocking Buffer for Fluorescent Western Blotting; BLOTTO; Bovine Serum Albumin (BSA); ELISA Microwell Blocking Buffer; Goat Serum; IPTG (isopropyl beta-D-thiogalactoside) Inducer; Normal Goat Serum (NGS); Normal Rabbit Serum; Normal Rat Serum; Normal Horse Serum; Normal Sheep Serum; Nitrophenyl phosphate buffer (NPP); and Revitablot™ Western Blot Stripping Buffer.

In some embodiments, the electrode comprises a nanocomposite coating comprising a mixture of a conducting element and a denatured proteinaceous material coated on at least a part of a surface, e.g. a conductive surface of the electrode. The proteinaceous material can be reversibly or non-reversibly denatured. In some embodiments, the proteinaceous material can be non-reversibly denatured. For example, the surface, e.g. the conductive surface of the electrode is at least partially coated with a nanocomposite coating comprising a conducting element mixed with denatured Bovine Serum Albumin (BSA).

In some embodiments, the nanocomposite coating comprises a three dimensional, porous matrix. In some embodiments, the nanocomposite coating comprises a pore density of about 70 pores μm⁻². In some embodiments, the nanocomposite coating comprises a pore density of about 10, 20, 30, 40, 50, 60 70, 80, 90, 100, 110, 120, 130, 140, or 150 pores μm⁻². In some embodiments, the nanocomposite coating comprises an average pore radius of about 30 nm. In some embodiments, the nanocomposite coating comprises an average pore radius of about 15, 20, 21, 22, 23, 24, 25, 26, 27, 28, 29, 30, 31, 32, 33, 34, 35, 36, 37, 38, 39, 40, 45, or 50 nm. In some embodiments, the nanocomposite coating comprises an average nearest-neighbor distance between pores of about 60 nm. In some embodiments, the nanocomposite coating comprises an average nearest-neighbor distance between pores of about 10, 20, 30, 40, 50, 60, 70, 80, 90, or 100 nm. In some embodiments, the nanocomposite coating comprises an average pore depth of about 4.3 nm. In some embodiments, the nanocomposite coating comprises an average pore depth of about 1, 1.5, 2, 2.5, 3, 3.5, 3.6, 3.7, 3.8, 3.9, 4, 4.1, 4.2, 4.3, 4.5, 4.6, 4.7, 4.8, 4.9, 5, 6, 7, 8, 9, or 10 nm. In some embodiments, the nanocomposite coating comprises a maximum pore depth of about 7.9 nm. In some embodiments, the nanocomposite coating comprises a maximum pore depth of about 1, 1.5, 2, 2.5, 3, 3.5, 4, 4.5, 5, 6, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.9, 9, or 10 nm.

The conducting element can comprise any conducting material known in the art. Further, the conducting element can be in any form. For example, the conducting element can be in form of particles, rods, fibers, nanoparticles and the like. In some embodiments, the conducting element comprises conductive and semi-conductive materials. For example, the conducting element comprises conductive and semi-conductive particles, rods, fibers, nano-particles and/or polymers.

In some embodiments, the conductive element comprises gold. For example, the conductive element comprises gold particles, rods, fibers, and/or nano-particles. Accordingly, in some embodiments, the electrode comprises a mixture of gold particles, rods, fibers, and/or nano-particles and a proteinaceous material coated on at least a part of a surface, e.g. a conductive surface of the electrode. For example, the surface, e.g. the conductive surface of the electrode is at least partially coated with a nanocomposite coating comprising gold particles, rods, fibers, and/or nano-particles mixed with denatured Bovine Serum Albumin.

In some embodiments, the conductive element comprises an allotrope of carbon atoms arranges in a hexagonal lattice. Thus, in some embodiments, the electrode comprises a mixture of an allotrope of carbon having atoms arranged in a hexagonal lattice and a proteinaceous material coated on at least a part of a surface, e.g. a conductive surface of the electrode. The proteinaceous material can be reversibly or non-reversibly denatured. In some embodiments, the proteinaceous material can be non-reversibly denatured. For example, the surface, e.g. the conductive surface of the electrode is at least partially coated with a nanocomposite coating comprising carbon nanotubes, graphene and/or reduced graphene oxide mixed with denatured Bovine Serum Albumin.

As used herein “proteinaceous” material includes proteins and peptides, functionalized proteins, copolymers including proteins, natural and synthetic variants of these, and mixtures of these. In some embodiments, the proteinaceous material is BSA.

As used herein, “denaturing” is the process of modifying the quaternary, tertiary and secondary molecular structure of a protein from its natural, original or native state. For example, such as by breaking weak bonds (e.g., hydrogen bonds), which are responsible for the highly ordered structure of the protein in its natural state. The process can be accomplished by, for example: physical means, such as by heating, sonication or shearing; by chemical means such as acid, alkali, inorganic salts and organic solvents (e.g., alcohols, acetone or chloroform); and by radiation. A denatured protein, such as an enzyme, losses its original biological activity. In some instances, the denaturing process is reversible, such that the protein molecular structure is regained by the re-forming of the original bonding interactions at least to the degree that the original biological function of the protein is restored. In other instances, the denaturing process is irreversible or non-reversible, such that the original and biological function of the protein is not restored. Cross-linking, for example after denaturing, can reduce or eliminate the reversibility of the denaturing process. Thus, in some embodiments, the proteinaceous material is cross-linked.

The degree of denaturing can be expressed as a percent of protein molecules that have been denatured, such as a mole percent. Some methods of denaturing can be more efficient than others. For example, under some conditions, sonication applied to BSA can denature about 30-40% of the protein and the denaturing is reversible. When BSA is denatured it undergoes two structural stages. The first stage is reversible whilst the second stage is irreversible (e.g., non-reversible) but does not necessarily result in a complete destruction of the ordered structure. For example, heating up to 65° C. can be regarded as the first stage, with subsequent heating above that as the second stage. At higher temperatures, further transformations are seen. In some embodiments, BSA is denatured by heating above about 65° C. (e.g., above about 70° C., above about 80° C., above about 90° C., above about 100° C., above about 110° C., above about 120° C.), below about 200° C. (below about 190° C., 180° C., 170° C., 160° C., 150° C.), and for at least about 1 minute (e.g., at least about 2, 3, 4, 5, 10 or 20 minutes) but less than about 24 hours (e.g., less than about 12, 10, 8, 6, 4, 2 1 hour). Embodiments include any ranges herein described, for example heating above about 90° C. but below about 150° C. and for at least 2 minutes but less than one hour.

In some embodiments the proteinaceous material used for coating the electrode is at least about 20% to about 100% (e.g., 20%, 30%, 40%, 50%, 60%, 70%, 80%, 90%, or 100%) denatured. In some embodiments, less than 50% of the denatured protein reverts back to its natural state (e.g., less than 40%, less than 30%, less than 20%, less than 10%, less than 1%). Therefore, the reversibility of the denaturing can be described as being 50% reversible, 40% reversible (60% irreversible), 30% reversible (70% irreversible), 20% reversible (80% irreversible), 10% reversible (90% irreversible) or even 0% reversible (100% irreversible).

Carbon nanotubes (CNTs) and graphene are allotropes of carbon with sp² carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder. The allotropes of carbon can include some functionalization, such as oxygen, carboxylates, epoxides, amines, amides and combinations of these, as described below. In some embodiments, graphene is reduced graphene oxide (rGO).

Reduced graphene oxide is prepared from reduction of graphene oxide by thermal, chemical or electrical treatments. For example, treating the graphene oxide with hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionalized rGO can be formed by a modified Buchere reaction, wherein ammonia and graphene oxide are reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %).

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration.

The carbon nanotubes, graphene oxide and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%, more than 99% of available carboxylic acid groups). In some embodiments the functionalization can be with a redox active compound or fragment (e.g., a metallocene, a viologen), antibody, a DNA strand, an RNA strand, a peptide, an antibody, an enzyme, a molecular receptor, a fragment of one of these or combination of these.

The allotropes of carbon having hexagonal lattices of carbon atoms, such as CNTs and rGO, can confer electroactivity (e.g., conductivity). Other conductive elements such as pure graphene, fullerenes, conductive and semi-conductive particles, rods, fibers and nano-particles (e.g., Gold), and conductive polymers (e.g., polypyrrole, polythiophene, polyaniline) can also be used to replace the CNTs and rGO or blended/combined with CNTs to modulate (e.g., improve) the conductivity, improve the stability and/or improve the stability of the coatings.

Exemplary coating materials for use are also described in PCT Patent Application No. PCT/US2018/04407, content of which is incorporated herein by reference in its entirety.

In some embodiments, the electrode can be comprised in an electrochemical sensor. Generally, the electrochemical sensor comprises a fluid-contact surface and an electrode described herein immobilized on at least a portion of the fluid-contact surface. The fluid-contact surface is a non-electrically conductive surface. Exemplary non-electrically conductive surfaces include, but are not limited to, plastic, poly(carbonate) (PC), poly(methyl methacrylate) (PMMA), cyclic olefin polymers (COP), cyclic olefin copolymers (COC), silicon nitride, parylene, kapton, styrene-ethylene-butylene-styrene (SEBS), poly-dimethylsiloxane (PDMS), polyimide, silicon dioxide, and any combination thereof.

In some embodiments, the fluid-contact surface further comprises a counter electrode, a reference electrode, a positive control electrode, a negative control electrode, or any combination thereof immobilized thereon

In some embodiments, the electrochemical sensor comprises one or more microfluidic flow cells. In some embodiments, the electrochemical sensor comprises one or more open wells. Some embodiments comprise both one or more microfluidic flow cells and one or more open wells.

In some embodiments, the electrochemical sensor comprises (i) an electrode described herein; (ii) a contact pad, which connects the electrodes (e.g., an electrode comprising an effector strand, control electrodes, reference electrodes, etc.) to a measuring unit (i.e., readout instrumentation); and (iii) a conductive track that links (i) to (ii). In general, (iii) is not exposed to fluid samples, (iii) can be covered by a polymer layer (e.g., SU-8) or simply hidden from the fluid sample using microfluidics.

### Effector Nucleic Acid Strand

The effector nucleic acid strand is a DNA or RNA oligonucleotide that can be cleaved by an activated CRISPR effector protein. The nucleotide sequence of the effector nucleic acid strand can be generic i.e. not the same as a target molecule.

The effector nucleic acid can be of any desired length. For example, the effector nucleic acid is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the effector nucleic acid is less than about 75, 50, 45, 40, 35, 30, 25, 20, 15, 12, or fewer nucleotides in length. Preferably the effector nucleic acid strand is 10-30 nucleotides in length.

In some embodiments of any one of the aspects, the effector strand is immobilized on a surface of the electrode, e.g., on the conductive surface of the electrode. For example, the effector strand can be covalently or non-covalently linked to a surface of the electrode.

For immobilizing on a surface of the electrode, e.g., on the conductive surface of the electrode, the effector strand can comprise a functional group for immobilization. It is noted that the functional group for immobilization can be located anywhere in the effector strand. For example, the functional group for immobilization can be at the 5′-end of the effector strand. Alternatively, the functional group for immobilization can be at the 3′-end of the effector strand. In some other non-limiting examples, the functional group for immobilization can be at an internal position of the effector strand.

In some embodiments, the effector strand comprises a functional group for conjugation with an electroactive label. The functional group for conjugation with an electroactive label can be placed anywhere in the effector strand. For example, the functional group for conjugation with an electroactive label can be at the 5′-end of the effector strand. Alternatively, the functional group for with an electroactive label can be at the 3′-end of the effector strand. In some other non-limiting examples, the functional group for conjugation with an electroactive label can be at an internal position of the effector strand.

In some embodiments, the effector nucleic acid can be conjugated with at least one electroactive label. The electroactive label can be placed anywhere in the effector strand. For example, the electroactive label can be at the 5′-end of the effector strand. Alternatively, the electroactive label can be at the 3′-end of the effector strand. In some other non-limiting examples, the electroactive label can be at an internal position of the effector strand.

In some embodiments, the effector nucleic acid strand comprises a functional group for immobilization on a surface of the electrodes and a functional group for conjugation with an electroactive label. The functional group for immobilization and the functional group for conjugation can be at opposing ends of the effector strand.

As disclosed herein, the effector strand can comprise a nucleic acid modification. For example, the effector strand can comprise a nucleic acid modification that inhibits or reduces cleavage of the effector strand by a nuclease, e.g., a CRISPR system effector protein.

In some embodiments, the effector strand comprises a peptide nucleic acid (PNA). For example, the effector strand is PNA.

### Detector Nucleic Acid Strand

The detector nucleic acid strand is a DNA or RNA oligonucleotide comprising a nucleotide sequence substantially complementary to the effector nucleic acid strand. For example, the degree of complementarity between the effector and detector nucleic acid strands, when optimally aligned using a suitable alignment algorithm, is about or more than about 50%, 60%, 75%, 80%, 85%, 90%, 95%, 97.5%, 99%), or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

Like the effector nucleic acid strand, the detector nucleic acid can be of any desired length. For example, the detector nucleic acid is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the detector nucleic acid is less than about 75, 50, 45, 40, 35, 30, 25, 20, 15, 12, or fewer nucleotides in length. Preferably the detector nucleic acid strand is 10-30 nucleotides in length.

In some embodiments, the effector strand comprises a functional group for conjugation with an electroactive label. The functional group for conjugation with an electroactive label can be placed anywhere in the effector strand. For example, the functional group for conjugation with an electroactive label can be at the 5′-end of the effector strand. Alternatively, the functional group for with an electroactive label can be at the 3′-end of the effector strand. In some other non-limiting examples, the functional group for conjugation with an electroactive label can be at an internal position of the effector strand.

In some embodiments, the detector nucleic acid can be conjugated with at least one electroactive label. The electroactive label can be placed anywhere in the detector strand. For example, the electroactive label can be at the 5′-end of the detector strand. Alternatively, the electroactive label can be at the 3′-end of the detector strand. In some other non-limiting examples, the electroactive label can be at an internal position of the detector strand.

In some embodiments, the detector strand comprises a functional group for conjugation with an electroactive label. The functional group for conjugation with an electroactive label can be placed anywhere in the detector strand. For example, the functional group for conjugation with an electroactive label can be at the 5′-end of the detector strand. Alternatively, the functional group for with an electroactive label can be at the 3′-end of the detector strand. In some other non-limiting examples, the functional group for conjugation with an electroactive label can be at an internal position of the detector strand.

As disclosed herein, the detector strand can comprise a nucleic acid modification. For example, the detector strand can comprise a nucleic acid modification that inhibits or reduces cleavage of the effector strand by a nuclease, e.g., a CRISPR system effector protein.

In some embodiments, the detector strand comprises a peptide nucleic acid (PNA). For example, the detector strand is fully PNA.

### Electroactive Label

An “electroactive label” is a molecule that is detectable on application of an electric field. Examples of an electroactive label include organic labels and organometallic labels. In one aspect the electroactive label includes a metallocene, including substituted metallocenes or a derivative thereof which is compatible with an aqueous environment. The metallocene can be, for example, ferrocene, cobaltocene or derivatives thereof. Substituted metallocenes such as halogen-substituted metallocenes, metallocene comprising an amide-substituted cyclopentadiene or other derivatives such as ansa-metallocenes, metallocenium cations such as ferrocenium, [Fe(C5H5)2]+, triple decker complexes (compounds with three Cp anions and two metal cations in alternating order, can also be used. Some exemplary metallocenes include, but are not limited to ferrocene, cobaltocene, nickelocene, ruthenocene, vanadocene, chromocene, decamethylmanganocene, decamethylrhenocene, tungstencense, titanocene, and zirconocene.

In some embodiments, the electroactive label includes quinines, nitro heterocycles, NAD+, NADP+, nitrogen-containing aromatics and heterocycle.

As used herein, the term “electroactive label” also encompasses molecules that produce a molecule that is detectable on application of an electric field. For example, the electroactive label can be an enzyme capable of generating or producing a molecule that is detectable on application of an electric field, for example, by changing the oxidation state of a substrate molecule.

In some embodiments, the electroactive label is horseradish peroxidase (HRP), alkaline phosphatase (AP), glucose oxidase (GOx), tyrosinase, urease, DNAzyme, aptazyme, or any combination thereof. In some preferred embodiments, the electroactive label is HRP.

In some embodiments, the electroactive label is a dsDNA intercalator, such as methylene blue.

### Mediator Composition

When the electroactive label is an enzyme, a composition comprising an electroactive mediator, i.e., a substrate for the enzyme is introduced onto the electrode, wherein a reaction of the electroactive mediator with the enzyme forms an electroactive precipitate locally adsorbed at the surface of the electrode. Then a voltage is applied to the electrode, wherein the voltage corresponds to the standard redox potential of the electroactive precipitate, and a current generated from the electrode is measured. Composition comprising an electroactive mediator is also referred to as electroactive mediator precipitating composition herein.

Accordingly, in some embodiments of any one of the aspects, the nucleic acid detection described herein further comprises an electroactive mediator precipitating composition

In some embodiment, the electroactive mediator precipitating composition comprises a substrate for the enzyme, e.g., the reporter enzyme. Exemplary reporter enzyme substrates include, but are not limited to, hydrogen peroxide, carbamide peroxide, nucleotides, oligonucleotides, RNA, DNA, phosphorylated peptides, phosphorylated proteins, phosphorylated small molecules, glucose, phenols, tyrosine, dopamine, catechol, urea, and any combination thereof. In some embodiments, the reporter enzyme substrate is hydrogen peroxide.

In some embodiments, the electroactive mediator precipitating composition comprises an electroactive mediator. Exemplary electroactive mediators include, but are not limited to, 3,3′,5,5′-tetramethylbenzidine (TMB), o-phenylenediamine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combinations thereof. In some preferred embodiments the electroactive mediator is TMB.

In some embodiments, the electroactive mediator precipitating composition further comprises a precipitating agent. The precipitating agent can be selected from the group consisting of a water-soluble polymer, a pyrrolidinone polymer, a polyaniline, a polypyrrole, a polythiophene, alginic acid, methyl vinyl ether/maleic anhydride copolymer, dextran sulfate, carrageenan, and any combinations thereof. In some preferred embodiments, the precipitating agent is a pyrrolidinone polymer.

In some embodiments, the electroactive mediator precipitating composition comprises an electroactive mediator and a substrate for the enzyme.

In some embodiments, the electroactive mediator precipitating composition comprises an electroactive mediator and a precipitating agent.

the electroactive mediator precipitating composition comprises a precipitating agent and a substrate for the enzyme.

In some embodiments, the electroactive mediator precipitating composition comprises an electroactive mediator, a substrate for the enzyme and a precipitating agent.

### CRISPR

Microbial Clustered Regularly Interspaced Short Palindromic Repeats (CRISPR) and CRISPR-associated (CRISPR-Cas) adaptive immune systems contain programmable endonucleases, such as Cas12a Cpf1 (also referred to as Cpf1) and Cas9. Although both Cas12a and Cas9 and target DNA, single effector RNA-guided RNases also have been recently discovered (Shmakov et al., 2015) and characterized (Abudayyeh et al., 2016; Smargon et al., 2017). These programmable endonucleases and RNases provide a platform for specific nucleic acid (DNA or RNA) sensing. RNA-guided endonucleases, such as Cas 12a, Cas14a, CasPhi, CasX, and Cas9, can be easily and conveniently reprogrammed using CRISPR guide RNA (gRNAs) to cleave target DNAs. RNA-guided RNases, such as C2c2, can be easily and conveniently reprogrammed using CRISPR RNA (crRNAs) to cleave target RNAs. See e.g., International Patent Publication WO 2020/142754, the content of which is incorporated by reference herein.

Once activated through recognition of the target DNA (e.g., single- or double-stranded DNA) or RNA, many of the CRISPR-Cas endonucleases and RNases exhibit promiscuous non-specific DNase or RNase activity. Thus, after cleavage of the target DNA (e.g., dsDNA) or RNA, the CRISPR-Cas endonucleases and RNases can lead to “collateral” cleavage of any non-targeted DNAs or RNAs present in proximity.

In general, a CRISPR-Cas or CRISPR system as used in herein refers collectively to transcripts and other elements involved in the expression of or directing the activity of CRISPR-associated (“Cas”) genes, including sequences encoding a Cas gene, a tracr (transactivating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas12a, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

The CRISPR-Cas effector protein can be from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methyl obacterium or Acidaminococcus.

In some embodiments, the effector protein can comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs can comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methyl obacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methyl obacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In the context of formation of a CRISPR complex, “target sequence” refers to a sequence to which a guide sequence is designed to have complementarity, where hybridization between a target sequence and a guide sequence promotes the formation of a CRISPR complex. A target sequence can be DNA or RNA. Generally, the term target nucleic acid refers to a polynucleotide being or comprising the target sequence. In other words, the target nucleic acid can be a polynucleotide or a part of a polynucleotide to which a part of the gRNA, i.e. the guide sequence, is designed to have complementarity and to which the effector function mediated by the complex comprising CRISPR effector protein and a gRNA is to be directed.

It is noted that the effector protein can be a DNA targeting CRISPR-Cas protein or an RNA targeting CRISPR-Cas protein. Exemplary CRISPR-Cas proteins include, but are not limited to, Cas1, Cas1B, Cas2, Cas3, Cas4, Cas5, Cash, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologues thereof, or modified versions thereof.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins can but need not be structurally related, or are only partially structurally related. An “orthologue” of a protein as used herein is a protein of a different species which performs the same or a similar function as the protein it is an orthologue of Orthologous proteins can but need not be structurally related, or are only partially structurally related. Homologs and orthologs can be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins can but need not be structurally related, or are only partially structurally related.

In some embodiments, the effector protein has a sequence homology or sequence identity of at least 60%, more particularly at least 70, such as at least 80%, more preferably at least 85%, even more preferably at least 90%, such as for instance at least 95%, with the wild-type sequence. The skilled person will understand that this includes truncated forms of effector protein whereby the sequence identity is determined over the length of the truncated form.

The CRISPR-Cas effector protein can be from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methyl obacterium or Acidaminococcus.

In some embodiments, the effector protein can be Cas 9, Cas12a, Cas13a, CasX, CasPhi or Cas14. In some embodiments, the effector protein is Cas12a, also known as Cpf1.

### Guide Nucleic Acid Strand

As used herein, the terms “guide nucleic acid,” “guide sequence,” “crRNA,” “guide RNA,” or “single guide RNA,” or “gRNA” refers to a polynucleotide comprising any polynucleotide sequence having sufficient complementarity with a target nucleic acid sequence to hybridize with the target nucleic acid sequence and to direct sequence-specific binding of a CRISPR complex comprising the guide sequence and a CRISPR effector protein to the target nucleic acid sequence.

In some example embodiments, the degree of complementarity, when optimally aligned using a suitable alignment algorithm, is about or more than about 50%, 60%, 75%, 80%, 85%, 90%, 95%, 97.5%, 99%), or more. Optimal alignment can be determined with the use of any suitable algorithm for aligning sequences. Exemplary algorithms for determining optimal alignment include, but are not limited to, the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

The guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a nucleic acid strand is less than about 75, 50, 45, 40, 35, 30, 25, 20, 15, 12, or fewer nucleotides in length. Preferably the guide nucleic acid sequence is 10-30 nucleotides long.

### Functional Group

It is noted that a functional group for conjugating an electroactive label to a nucleic acid strand or immobilizing a nucleic strand on a surface of the electrode can be any functional group that can react with another molecule or functional group and form a covalent or non-covalent linkage. Exemplary functional groups include, but are not limited to, acetal, acetylene, acid amide, acid anhydride, acid imide, alcohol, aldehyde, allene, amidine, amine or amino, aminooxy, azanol, azide, azo-compound, azoxy compound, carbamate, carbodiimides, carboxylic acid, cyanate, cyanide, diazo, diazol, disulfide, enamine, epoxy, ester, ether, halide, hydrazide, hydrazine, hydrazone, hydroxamic acid, hydroxyl, imide ester, imines, isocyanate, isonitrile, isothiocyanate, ketal, ketone, mercaptan, nitrile, nitro, nitrone, nitroso, ortho esters, oxide, oxime, phenol, phosphate group, pseudo-urea, semicarbazide, sulfenic acid, sulfide, sulfinic acid, sulfite, sulfone, sulfonic acid, sulfoxide, sulfuric ester, sulphur hydroxamic acid, thiocyanate, thiol, urea, and ynamine.

In some embodiments, the functional group can be one member of a binding pair. A “binding pair”, “coupling molecule pair” and “coupling pair” are used interchangeably and without limitation herein to refer to the first and second molecules or functional groups that specifically bind to each other. For example, the binding can be through one or more of a covalent bond, a hydrogen bond, an ionic bond, and a dative bond. In some embodiments one member of the binding pair is conjugated with a solid substrate while the second member is conjugated with the linker. A binding pair can be used for linking the linker to the substrate, and/or for linking the linker to the analyte-related molecule.

Exemplary coupling molecule pairs also include, without limitations, any haptenic or antigenic compound in combination with a corresponding antibody or binding portion or fragment thereof (e.g., digoxigenin and anti-digoxigenin; mouse immunoglobulin and goat anti-mouse immunoglobulin) and non-immunological binding pairs (e.g., biotin-avidin, biotin-streptavidin), hormone (e.g., thyroxine and cortisol-hormone binding protein), receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes). The coupling molecule pair can also include a first molecule that is negatively charged and a second molecule that is positively charged.

One example of using coupling pair conjugation is the biotin-avidin or biotin-streptavidin conjugation. In this approach, one of the members of the coupling pair is biotinylated and the other is conjugated with avidin or streptavidin. Many commercial kits are also available for biotinylating molecules. For example, an aminooxy-biotin (AOB) can be used to covalently attach biotin to a molecule with an aldehyde or ketone group. In some embodiments, the functional group is biotin or a variant thereof.

One non-limiting example of using conjugation with a coupling molecule pair is the biotin-sandwich method. See, e.g., Davis et al., 103 PNAS 8155 (2006). The two molecules to be conjugated together are biotinylated and then conjugated together using tetravalent streptavidin. In addition, a peptide can be coupled to the 15-amino acid sequence of an acceptor peptide for biotinylation (referred to as AP; Chen et al., 2 Nat. Methods 99 (2005)). The acceptor peptide sequence allows site-specific biotinylation by the E. coli enzyme biotin ligase (BirA; Id.). An engineered microbe surface-binding domain can be similarly biotinylated for conjugation with a solid substrate. Many commercial kits are also available for biotinylating proteins. Another example for conjugation to a solid surface would be to use PLP—mediated bioconjugation. See, e.g., Witus et al., 132 JACS 16812 (2010).

Other examples for forming a coupling pair include click chemistry. As used herein “click chemistry” refers to a class of small molecule reactions which can be used for the linking of a binding pair and is not a single specific reaction but rather describes the method of generating products by mimicking nature which produces substance by joining of small modular units. Although useful for biochemical reactions, click chemistry is not limited to biological conditions. Click reactions are efficient and easy to used, occurring in one pot without any special precautions against water and air, do not produce offensive (e.g., not toxic) byproducts, and, because they are characterized by a high thermodynamic driving force that drives the reaction quickly to a single reaction product, require minimal or no final isolation and purification. Examples of click chemistry includes the copper-catalyzed reaction of an azide with an alkyne to form a 5-membered heteroatom ring (e.g., a Cu(I)-catalyzed azide-alkyne cycloaddition), the thiol-Michael Addition reaction such as reaction of a thiol group with a maleimide group, strain-promoted azide-alkyne cycloaddition, strain-promoted alkyne-nitrone cycloaddition, reactions of strained alkenes, alkene and azide [3+2] cycloaddition, alkene and tetrazine inverse-demand Diels-Alder, and alkene and tetrazole photoclick reaction.

### Target Nucleic Acid

In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence can comprise any polynucleotide, such as DNA or RNA polynucleotides. In some embodiments, a target sequence is located in the nucleus or cytoplasm of a cell, and can include nucleic acids in or from mitochondrial, organelles, vesicles, liposomes or particles present within the cell. The target sequence can be any desired nucleic acid. Further, the target sequence can be naturally occurring or synthetic nucleic acid. Thus, in some embodiments, the target sequence is a naturally occurring nucleic acid. A naturally occurring sequence includes a nucleic acid isolated and/or purified from a natural source.

The target sequence can be within a double-stranded or single-stranded region of the target. In some embodiments, the target sequence can be a sequence within a DNA molecule. The target DNA molecule can be genomic DNA, cell free DNA (cfDNA), mitochondrial DNA, cDNA or the like. In some embodiments, the target sequence can be a sequence within an RNA molecule. The RNA molecule can be messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), or small cytoplasmic RNA (scRNA). It is noted that the target nucleic acid can be a naturally occurring nucleic acid or a synthetic nucleic acid.

In some embodiments of the various aspects described herein, the target sequence is from an organism, including but not limited to a prokaryote, eukaryote, archaeabacteria, animal, plant, protist, parasite, fungus, or bacterium. In some embodiments of the various aspects described herein, the target sequence is from a virus. In some embodiments of the various aspects described herein, the target sequence is from a human. In some embodiments of the various aspects described herein, the target sequence is from a pathogenic organism. In some embodiments of the various aspects described herein, the target sequence is from a non-pathogenic organism.

In some embodiments of the various aspects described herein, the target sequence is from a bacterium, which can be a pathogenic or non-pathogenic bacterial species. Non-limiting examples of pathogenic bacteria that can comprise the target sequence include spirochetes (e.g. Borrelia), actinomycetes (e.g. Actinomyces), mycoplasmas, Rickettsias, Gram negative aerobic rods, Gram negative aerobic cocci, Gram negatively facultatively anaerobic rods (e.g. Erwinia and Yersinia), Gram-negative cocci, Gram negative coccobacilli, Gram positive cocci (e.g. Staphylococcus and Streptococcus), endospore-forming rods, and endospore-forming cocci.

Non-limiting examples of bacterial pathogens include Bacillus, Brucella, Burkholderia, Francisella, Yersinia, Streptococcus, Haemophilus, Nisseria, Listeria, Clostridium, Klebsiella, Legionella, Escherichia (e.g., E. coli), Mycobacterium, Staphylococcus, Campylobacter, Vibrio, and Salmonella, as well as drug and multidrug resistant strains and highly virulent strains of these pathogenic bacteria. Non-limiting examples of known food-borne bacterial pathogens include Salmonella, Clostridium, Campylobacter spp., Staphylococcus, Salmonella, Escherichia (e.g., E. coli), and Listeria. In some embodiments, non-limiting examples of bacterial pathogens include Bacillus anthracis, Brucella abortus, Brucella melitensis, Brucella suis, Burkholderia mallei, Burkholderia pseudomallei, Francisella tularensis, Yersinia pestis, Streptococcus Group A and B, MRSA, Streptococcus pneumonia, Haemophilus influenza, Nisseria meningitides, Listeria monocytegenes, Clostridium difficile, Klebsiella, highly virulent pathogenic strains of E. coli, Mycobacterium tuberculosis, Staphylococcus aureus, Campylobacter spp, Salmonella spp, and Clostridium perfringens, as well as drug and multidrug resistant strains and highly virulent strains of these pathogenic bacteria. In some embodiments, non-limiting examples of known food-borne bacterial pathogens include Salmonella, non typhoidal Clostridium perfringens, Campylobacter spp., Staphylococcus aureus, Salmonella, nontyphoidal, Campylobacter spp., E. coli (STEC) 0157, and Listeria monocytogenese. In some embodiments of the various aspects described herein, the target sequence is from a Borrelia bacterial species, such as Borrelia burgdorferi.

In some embodiments of the various aspects described herein, the target sequence is from a fungus, which can be a pathogenic or non-pathogenic fungal species. Non-limiting examples of fungi that can comprise the target sequence include yeast and molds, such as Aspergillus, Cladosporium, Epicoccum, Penicillium, Acremonium, Exophiala, Phialophora, Trichoderma, Fusarium, Phoma, Mucorales, Geotrichum, Candida, and Claviceps.

In some embodiments of the various aspects described herein, the target nucleic acid is a viral DNA or RNA. For example, the target nucleic acid is from an RNA virus.

In some embodiments, the RNA virus is Group III (i.e., double stranded RNA (dsRNA)) virus. In some embodiments of the various aspects described herein, the Group III RNA virus belongs to a viral family selected from the group consisting of: Amalgaviridae, Birnaviridae, Chrysoviridae, Cystoviridae, Endornaviridae, Hypoviridae, Megabirnaviridae, Partitiviridae, Picobirnaviridae, Reoviridae (e.g., Rotavirus), Totiviridae, Quadriviridae. In some embodiments of the various aspects described herein, the Group III RNA virus belongs to the Genus Botybirnavirus. In some embodiments of the various aspects described herein, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments of the various aspects described herein, the RNA virus is a Group IV (i.e., positive-sense single stranded (ssRNA)) virus. In some embodiments of the various aspects described herein, the Group IV RNA virus belongs to a viral order selected from the group consisting of: Nidovirales, Picornavirales, and Tymovirales. In some embodiments of the various aspects described herein, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some embodiments of the various aspects described herein, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of the various aspects described herein, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of the various aspects described herein, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments of the various aspects described herein, the RNA virus is a Group V (i.e., negative-sense ssRNA) virus. In some embodiments of the various aspects described herein, the Group V RNA virus belongs to a viral phylum or subphylum selected from the group consisting of: Negarnaviricota, Haploviricotina, and Polyploviricotina. In some embodiments of the various aspects described herein, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments of the various aspects described herein, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of the various aspects described herein, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments of the various aspects described herein, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments of the various aspects described herein, the RNA virus is a Group VI RNA virus, which comprise a virally encoded reverse transcriptase. In some embodiments of the various aspects described herein, the Group VI RNA virus belongs to the viral order Ortervirales. In some embodiments of the various aspects described herein, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some embodiments of the various aspects described herein, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus).

In some embodiments of the various aspects described herein, the RNA virus is selected from influenza virus, human immunodeficiency virus (HIV), severe acute respiratory syndrome coronavirus 2 (SARS-CoV-2), and SARS-associated coronavirus (SARS-CoV). In some embodiments of the various aspects described herein, the RNA virus is influenza virus. In some embodiments of the various aspects described herein, the RNA virus is immunodeficiency virus (HIV). In some embodiments of the various aspects described herein, the RNA virus is severe acute respiratory syndrome coronavirus 2 (SARS-CoV-2). In some embodiments of the various aspects described herein, the RNA virus is SARS-associated coronavirus (SARS-CoV). In some embodiments of the various aspects described herein, the RNA virus is any known RNA virus.

In some embodiments of the various aspects described herein, the viral RNA is an RNA produced by a virus with a DNA genome, i.e., a DNA virus. As a non-limiting example the DNA virus is a Group I (dsDNA) virus, a Group II (ssDNA) virus, or a Group VII (dsDNA-RT) virus. In some embodiments of the various aspects described herein, the RNA produced by a DNA virus comprises an RNA transcript of the DNA genome.

### Nucleic Acid Modifications

The guide nucleic acid, the effector strand and the detector strand can independently comprise one or more nucleic acid modifications known in the art. For example, the guide nucleic acid, the effector strand and/or the detector strand can independently comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs can be modified at the ribose, phosphate, and/or base moiety.

Exemplary nucleic acid modifications include, but are not limited to, nucleobase modifications, sugar modifications, inter-sugar linkage modifications, conjugates (e.g., ligands), and combinations thereof. In one embodiment, a modification does not include replacement of a ribose sugar with a deoxyribose sugar as occurs in deoxyribonucleic acid. Nucleic acid modifications are known in the art, see, e.g., US20160367702; US20190060458; U.S. Pat. Nos. 8,710,200; and 7,423,142, which are incorporated herein by reference in their entireties.

Exemplary modified nucleobases include, but are not limited to, thymine (T), inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3 carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH2-C(═O)—N(H)-5′) and amide-4 (3′-CH2-N(H)—C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH2-O-5′), formacetal (3 ′—O—CH2-O-5′), oxime, methyleneimino, methykenecarbonylamino, methylenemethylimino (MMI, 3′-CH2-N(CH3)-O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH2-S—C5′, C3′-O—P(O)—O—SS—C5′, C3′-CH2-NH—NH—C5′, 3′-NHP(O)(OCH3)-O-5′ and 3′-NHP(O)(OCH3)-O-5′

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

### Amplification of Target

The target nucleic acid (DNA or RNA) can be amplified prior to activating the CRISPR effector protein. Any suitable RNA or DNA amplification technique can be used. In certain example embodiments, the RNA or DNA amplification is an isothermal amplification. In certain example embodiments, the isothermal amplification can be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods can be used which include, but are not limited to, PCR, multiple displacement amplification (MDA), rolling circle amplification (RCA), ligase chain reaction (LCR), or ramification amplification method (RAM). A reverse-transcriptase step can also be used prior to amplification. For Example, the pre-amplification can include RT-LAMP, RT-NASBA, RT-RPA, RT-RCA or the like. The assay can also be coupled to transcription, e.g. T7 transcription.

In some embodiments, detection of a DNA target with the methods or systems described herein requires transcription of the DNA, amplified or non-amplified, into RNA prior to detection.

Accordingly, in some embodiments, the systems disclosed herein can include amplification reagents. Different components or reagents useful for amplification of nucleic acids include, but are not limited to, buffers, salts, nucleotide triphosphates, polymerases, primers and the like. In some embodiments, the system can also include cell lysis reagents in order to break open or lyse a cell for analysis of the materials therein.

### Detection Assay

In another aspect, the disclosure provides a method for detecting target nucleic acid molecules. As used herein, the term “detect” includes identifying the presence or absence of a target nucleic acid, and can also include quantifying the amount and/or concentration of a target nucleic acid in a sample.

Generally, the assay comprises contacting a sample suspected of comprising the target nucleic acid with a detection CRISPR system, wherein the CRISPR detection system comprises an effector protein and one or more guide nucleic acid (gNA) strands designed to bind to the target nucleic acid molecules. Contacting can comprise adding the CRISPR detection system to the sample. Alternatively, contacting can comprise adding the sample to a volume comprising the CRISPR detection system.

The sample and the CRISPR system are incubated for a period time. Incubation time is sufficient to allow the guide nucleic acid strand to hybridize with the target nucleic acid sequence and form a CRISPR complex comprising the guide strand, the target nucleic acid, and a CRISPR effector protein. Incubation time can be 120 minutes or less. For example, incubation time can be 2 hours, 1 hour, 30 minutes, 25 minutes, 20 minutes, 15 minutes, 10 minutes, 5 minutes, 2.5 minutes, 1 minute or less. In some embodiments, incubation time can be 15 minutes, 30 minutes, 60 minutes, 1.5 hours, 2 hours, 3 hours, or more. In some embodiments, the incubation time can be 1 minute or longer. As a non-limiting example, the incubation time can be at least 1 minute, at least 2 minutes, at least 3 minutes, at least 4 minutes, at least 5 minutes, at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, or at least 120 minutes.

The sample comprising the CRISPR complex is then contacted with the electrode. It is noted that the incubating the CRISPR system with the target sample can be done in the presence of the electrode. For example, the electrode can be present in an electrochemical cell or sensor. The sample and the CRISPR system can be added to the electrochemical cell or the sensor.

After a sufficient period of time to allow the CRISPR effector protein to cleave the effector strand on the electrode, the electrode is washed, e.g., with a buffer solution to remove any cleaved product from the electrode. The time period for cleavage can be a minute or more. For example, time for cleavage can be 5 minute, 10 minutes, 15 minutes, 30 minutes, 45 minutes, 60 minutes, 1.5 hours, 2 hours, 3 hours or more. In some embodiments, time period for cleavage 3 hours, 2 hours, 1.5 hours, 60 minutes, 45 minutes, 30 minutes, 15 minutes, 10 minutes, 5 minutes or less.

After the washing step, a voltage is applied to the electrode and a current generated from electrode is measured.

If the effector strand does not comprise an electroactive label, the method further comprises conjugating the effector strand with an electro active label and/or introducing a detector strand to the electrode, where the detector strand comprises an electroactive label. In some embodiments, the effector strand does not comprise an electroactive label and the method further comprises conjugating the effector strand with an electroactive label.

In some embodiments, the effector strand does not comprise an electroactive label and the method further introducing a detector strand to the electrode. After the detector strand has sufficient time for hybridizing with the effector strand on the electrode, the electrode can be washed to remove any unbound detector strands. The time period for hybridization can be a minute or more. For example, time for hybridization can be 5 minute, 10 minutes, 15 minutes, 30 minutes, 45 minutes, 60 minutes, 1.5 hours, 2 hours, 3 hours or more. In some embodiments, time period for cleavage 3 hours, 2 hours, 1.5 hours, 60 minutes, 45 minutes, 30 minutes, 15 minutes, 10 minutes, 5 minutes or less.

In some embodiments, the assay comprises contacting a sample suspected of comprising the target nucleic acid with a detection CRISPR system, wherein the CRISPR detection system comprises an effector protein and one or more guide nucleic acid (gNA) strands designed to bind to the target nucleic acid molecules. Contacting can comprise adding the CRISPR detection system to the sample. Alternatively, contacting can comprise adding the sample to a volume comprising the CRISPR detection system.

The sample and the CRISPR system are incubated for a period time. Incubation time is sufficient to allow the guide nucleic acid strand to hybridize with the target nucleic acid sequence and form a CRISPR complex comprising the guide strand, the target nucleic acid, and a CRISPR effector protein. Incubation time can be 30 minutes or less. For example, incubation time can be 25 minutes, 20 minutes, 15 minutes, 10 minutes, 5 minutes, 2.5 minutes, 1 minute or less.

The sample comprising the CRISPR complex is then contacted with a detector strand, optionally comprising an electroactive label. It is noted that the incubating the CRISPR system with the target sample can be done in the presence of the detector strand. For example, the CRISPR system, the target nucleic acid and the detector strands can be incubated together in one reaction vessel.

After a sufficient period of time to allow the CRISPR effector protein to cleave the detector strand, the mixture comprising the CRISPR system, the target nucleic acid and the cleaved, or at least partially cleaved, detector strands is added to the electrode. The time period for cleavage can be a minute or more. For example, time for cleavage can be 5 minute, 10 minutes, 15 minutes, 30 minutes, 45 minutes, 60 minutes, 1.5 hours, 2 hours, 3 hours or more. In some embodiments, time period for cleavage 3 hours, 2 hours, 1.5 hours, 60 minutes, 45 minutes, 30 minutes, 15 minutes, 10 minutes, 5 minutes or less.

In some embodiments, the electrode comprises an effector strand immobilized on a surface of the electrode. In such embodiments, the detector strand in the mixture is allowed to hybridize with the effector strand immobilized on the electrode. After a sufficient period of time to allow any detector strand in the mixture to hybridize with the effector strand immobilized on the electrode, the electrode can be washed, e.g., with a buffer solution to remove unbound nucleic acid strands and/or CRISPR components from the electrode. After the washing step, a voltage is applied to the electrode and a current generated from electrode is measured. If the detector strand does not comprise an electroactive label, the method further comprises conjugating the detector strand with an electroactive label. The conjugation can be prior to or after hybridizing the detector strand with the effector strand.

In some embodiments, the electrode does not comprise an effector strand immobilized on a surface of the electrode. In such embodiments, the method comprises immobilizing the detector strand on a surface of the electrode. After immobilizing the detector strand, the electrode can be washed, e.g., with a buffer solution to remove unbound nucleic acid strands and/or CRISPR components from the electrode. After the washing step, a voltage is applied to the electrode and a current generated from electrode is measured. If the detector strand does not comprise an electroactive label, the method further comprises conjugating the detector strand with an electroactive label. The conjugation can be prior to or after immobilizing the detector strand on the electrode.

In some embodiments, measuring the voltage comprises introducing an electroactive mediator precipitating composition onto electrode, wherein a reaction of the electroactive mediator precipitating composition with the electroactive label forms an electroactive precipitate at the surface of the electrode.

### Sample

In some embodiments, appropriate samples for use in the methods disclosed herein include a sample comprising a synthetic nucleic acid, in other words a nucleic acid that does not derive from any organism. In some embodiments, appropriate samples for use in the methods disclosed herein include any conventional biological sample suspected of comprising a target nucleic acid and obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacterium or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art. See, for example Schluger et al, J. Exp. Med. 176: 1327-33 (1992); Bigby et al, Am. Rev. Respir. Dis. 133:515-18 (1986); Kovacs et al, NEJM318:589-93 (1988); and Ognibene et al, Am. Rev. Respir. Dis. 129:929-32 (1984).

In other embodiments, a sample may be an environmental sample suspected of comprising a target nucleic acid, such as water, soil, or a surface such as industrial or medical surface.

In some embodiments, the method further comprises a step of extracting a nucleic acid from a sample. For example, extracting the DNA from a biological sample. Methods of extracting nucleic acids from sample, e.g., biological sample are well known in the art and could be used without modifications.

The systems and methods disclosed herein can also be used to screen environmental samples for contaminants by detecting the presence of target nucleic acids.

The low cost and adaptability of the assay platform lends itself to a number of applications including, but not limited to, rapid and sensitive detection of target nucleic acids in both clinical and environmental samples, general RNA/DNA quantitation, and rapid, multiplexed RNA/DNA expression detection. Additionally, the methods and systems disclosed herein can be adapted for detection of transcripts within biological settings, such as cells. Given the highly specific nature of the CRISPR effectors proteins, it is possible to track allelic specific expression of transcripts or disease-associated mutations in live cells

In some embodiments, the systems and methods, disclosed herein are directed to detecting the presence of one or more microbial agents in a sample, such as a biological sample obtained from a subject. The microbe can be a bacterium, a fungus, a yeast, a protozoan, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g. bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein can be used as guide therapeutic regimens, such as a selection of the appropriate antibiotic or antiviral. The embodiments disclosed herein can also be used to screen environmental samples (air, water, surfaces, food etc.) for the presence of microbial contamination.

The systems and methods described herein can distinguish between single nucleotide polymorphisms (SNPs). Accordingly, in some embodiments, the systems and methods, disclosed herein are directed to detecting the presence of an SNP.

As demonstrated herein, the methods and systems disclosed herein are capable of detecting down to attomolar concentrations of target molecules. Due to the sensitivity, a number of applications that require rapid and sensitive detection can benefit from the methods and systems disclosed herein, and are contemplated to be within the scope of the invention.

Also provided herein kits for detection of target nucleic acids. Generally, the kit comprises one or more components of the systems described herein.

Also provided herein are compositions comprising one or more components of the systems described herein.

Some exemplary embodiments of the various aspects described herein can be defined as follows:

Embodiment 1: A nucleic acid detection system comprising: (a) a detection CRISPR system comprising an effector protein and one or more guide nucleic acid strands designed to bind to corresponding target nucleic acid molecules; (b) an effector nucleic acid strand, optionally conjugated with at least one electroactive label; (c) an electrode comprising: (i) a conductive surface; and (ii) a nanocomposite coating comprising a mixture of a conducting element and a denatured proteinaceous material coated on at least a part of said conductive surface, wherein the effector nucleic acid strand is optionally immobilized on the conductive surface of the electrode; and (d) optionally, a detector nucleic acid strand substantially complementary to the effector nucleic acid strand and optionally conjugated with at least one electroactive label.

Embodiment 2: The nucleic acid detection system of Embodiment 1, wherein the CRISPR system effector protein is selected from the group consisting of Cas1, Cas1B, Cas2, Cas3, Cas4, Cas5, Cash, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and homologues thereof, or modified versions thereof.

Embodiment 3: The nucleic acid detection system of Embodiment 1 or 2, wherein the CRISPR system effector protein is selected from the group consisting of Cas 9, Cas12a, Cas13a, and Cas14.

Embodiment 4: The nucleic acid detection system of any one of Embodiments 1-3, wherein the effector protein is Cas12a.

Embodiment 5: The nucleic acid detection system of any one of Embodiments 1-4, wherein the CRISPR system effector protein is a DNA targeting protein.

Embodiment 6: The nucleic acid detection system of any one of Embodiments 1-5, wherein the CRISPR system effector protein is an RNA targeting protein.

Embodiment 7: The nucleic acid detection system of any one of Embodiments 1-6, wherein the effector strand comprises a functional group for immobilization on the conductive surface.

Embodiment 8: The nucleic acid detection system of Embodiment 7, wherein the functional group for immobilization is at the 5′-end of the effector strand.

Embodiment 9: The nucleic acid detection system of Embodiment 7, wherein the functional group for immobilization is at the 3′-end of the effector strand.

Embodiment 10: The nucleic acid detection system of any one of Embodiments 1-9, wherein the effector strand is immobilized on the conductive surface.

Embodiment 11: The nucleic acid detection system of any one of Embodiments 1-10, wherein the effector strand comprises a functional group for conjugating with at least one electroactive label.

Embodiment 12: The nucleic acid detection system of Embodiment 11, wherein the functional group for conjugating with the electroactive label is at the 5′-end of the detector strand.

Embodiment 13: The nucleic acid detection system of Embodiment 11, wherein the functional group for conjugating with the electroactive label is at the 3′-end of the detector strand.

Embodiment 14: The nucleic acid detection system of any one of Embodiments 1-13, wherein the effector strand comprises at least one electroactive label.

Embodiment 15: The nucleic acid detection system of any one of Embodiments 1-14, wherein the effector strand comprises a nucleic acid modification.

Embodiment 16: The nucleic acid detection system of any one of Embodiments 1-15, wherein the effector strand comprises a nucleic acid modification that inhibits or reduces cleavage of the effector strand by the CRISPR system effector protein.

Embodiment 17: The nucleic acid detection system of Embodiment 16, wherein the nucleic acid modification that inhibits or reduces cleavage of the effector strand by the CRISPR system effector protein is a peptide nucleic acid (PNA).

Embodiment 18: The nucleic acid detection system of any one of Embodiments 1-17, wherein the detector strand comprises a functional group for conjugating with the at least one electroactive label.

Embodiment 19: The nucleic acid detection system of Embodiment 18, wherein the functional group for conjugating with the electroactive label is at the 5′-end of the detector strand.

Embodiment 20: The nucleic acid detection system of Embodiment 18, wherein the functional group for conjugating with the electroactive label is at the 3′-end of the detector strand.

Embodiment 21: The nucleic acid detection system of any one of Embodiments 1-20, wherein the detector strand comprises at least one electroactive label.

Embodiment 22: The nucleic acid detection system of any one of Embodiments 1-21, wherein the detector strand comprises a nucleic acid modification.

Embodiment 23: The nucleic acid detection system of any one of Embodiments 1-22, wherein the detector strand comprises a nucleic acid modification that inhibits or reduces cleavage of the detector strand by the CRISPR system effector protein.

Embodiment 24: The nucleic acid detection system of Embodiment 23, wherein the nucleic acid modification that inhibits or reduces cleavage of the detector strand by the CRISPR system effector protein is a peptide nucleic acid

Embodiment 25: The nucleic acid detection system of any one of Embodiments 1-24, wherein the electrode is comprised in an electrochemical cell.

Embodiment 26: The nucleic acid detection system of any one of Embodiments 1-25, wherein the proteinaceous material is non-reversibly denatured.

Embodiment 27: The nucleic acid detection system of any one of Embodiments 1-26, wherein the proteinaceous material is cross-linked.

Embodiment 28: The nucleic acid detection system of any one of Embodiments 1-27, wherein the conducting element comprises conductive and semi-conductive particles, rods, fibers, nano-particles or polymers

Embodiment 29: The nucleic acid detection system of any one of Embodiments 1-28, wherein the conducting element comprises gold.

Embodiment 30: The nucleic acid detection system of any one of Embodiments 1-27, wherein the conducting element comprises an allotrope of carbon atoms arranged in a hexagonal lattice.

Embodiment 31: The nucleic acid detection system of Embodiment 30, wherein the allotrope of carbon is a functionalized material.

Embodiment 32: The nucleic acid detection system of Embodiment 30, wherein the allotrope of carbon is carbon nanotubes, reduced graphene oxide or mixtures thereof.

Embodiment 33: The nucleic acid detection system of Embodiment 32, wherein the carbon nanotube is carboxylated carbon nanotubes (CNTs) or aminated carbon nanotubes

Embodiment 34: The nucleic acid detection system of Embodiment 32, wherein the reduced graphene oxide is a carboxylated reduced graphene oxide or an aminated reduced graphene oxide.

Embodiment 35: The nucleic acid detection system of any one of Embodiments 1-34, wherein the electroactive label comprises an enzyme or a metallocene.

Embodiment 36: The nucleic acid detection system of any one of Embodiments 1-35, wherein the electroactive label comprises an enzyme, e.g., a reporter enzyme.

Embodiment 37: The nucleic acid detection system of Embodiment 36, wherein the enzyme is horseradish peroxidase (HRP), alkaline phosphatase (AP), glucose oxidase (GOx), tyrosinase, urease, a DNAzyme, an aptazyme, or any combinations thereof.

Embodiment 38: The nucleic acid detection system of Embodiment 37, wherein the enzyme is HRP.

Embodiment 39: The nucleic acid detection system of any one of Embodiments 1-35, wherein the electroactive label comprises a metallocene.

Embodiment 40: The nucleic acid detection system of Embodiment 39, wherein the metallocene is ferrocene, cobaltocene, nickelocene, ruthenocene, vanadocene, chromocene, decamethylmanganocene, decamethylrhenocene, tungstencense, titanocene, or zirconocene.

Embodiment 41: The nucleic acid detection system of any one of Embodiments 1-40, wherein the system further comprises an electroactive mediator precipitating composition.

Embodiment 42: The nucleic acid detection system of any one of Embodiments 1-41, wherein the system further comprises an electroactive mediator precipitating composition comprising a reporter enzyme substrate.

Embodiment 43: The nucleic acid detection system of Embodiment 42, wherein the reporter enzyme substrate is selected from the group consisting of hydrogen peroxide, carbamide peroxide, nucleotides, oligonucleotides, RNA, DNA, phosphorylated peptides, phosphorylated proteins, phosphorylated small molecules, glucose, phenols, tyrosine, dopamine, catechol, urea, and any combination thereof.

Embodiment 44: The nucleic acid detection system of Embodiment 43, wherein the reporter enzyme substrate is hydrogen peroxide.

Embodiment 45: The nucleic acid detection system of any one of Embodiments 1-44, wherein the system further comprises an electroactive mediator precipitating composition comprising an electroactive mediator.

Embodiment 46: The nucleic acid detection system of Embodiment 45, wherein the electroactive mediator is selected from the group consisting of 3,3′,5,5′-tetramethylbenzidine (TMB), o-phenylenedi amine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof.

Embodiment 47: The nucleic acid detection system of Embodiment 46, wherein the electroactive mediator is TMB.

Embodiment 48: Embodiment The nucleic acid detection system of Embodiment 41, wherein the electroactive mediator precipitating composition comprises a reporter enzyme substrate and an electroactive mediator.

Embodiment 49: The nucleic acid detection system of any one of Embodiments 1-48, wherein the system further comprises an electroactive mediator precipitating composition comprising a precipitating agent.

Embodiment 50: The nucleic acid detection system of Embodiment 49, wherein the precipitating agent is selected from the group consisting of a water-soluble polymer, a pyrrolidinone polymer, a polyaniline, a polypyrrole, a polythiophene, alginic acid, methyl vinyl ether/maleic anhydride copolymer, dextran sulfate, carrageenan, and any combination thereof.

Embodiment 51: The nucleic acid detection system of Embodiment 50, wherein the precipitating agent is a pyrrolidinone polymer.

Embodiment 52: The nucleic acid detection system of any one of Embodiments 1-51, wherein the system further comprises an electroactive mediator precipitating composition comprising a reporter enzyme substrate, an electroactive mediator, and a precipitating agent.

Embodiment 53: The nucleic acid detection system of any one of Embodiments 1-52, wherein the system further comprises a target nucleic acid.

Embodiment 54: The nucleic acid detection system of any one of Embodiments 1-53, wherein the nanocomposite coating comprises porous matrix, e.g., a three dimensional, porous matrix.

Embodiment 55: A method for detecting a nucleic acid, the method comprising: (a) contacting a target nucleic acid with a detection CRISPR system, wherein the CRISPR detection system comprises an effector protein and one or more guide nucleic acid strands designed to bind to or hybridize with the target nucleic acid molecules; (b) contacting the CRISPR detection system from (a) with an electrode, wherein the electrode comprises: (i) a conductive surface, (ii) a nanocomposite coating comprising a mixture of a conducting element and a denatured proteinaceous material coated on at least a part of said conductive surface, and (iii) an effector nucleic acid strand immobilized on the conductive surface; (c) contacting the electrode with a detector nucleic acid strand, wherein the detector nucleic acid stand is substantially complementary to the effector nucleic acid strand and is conjugated with at least one electroactive label; and (d) applying a voltage to the electrode and measuring a current generated from electrode.

Embodiment 56: A method for detecting a nucleic acid, the method comprising: (a) contacting a target nucleic acid with a detection CRISPR system, wherein the CRISPR detection system comprises an effector protein, one or more guide nucleic acid strands designed to bind to or hybridize with the target nucleic acid molecules, and a detector nucleic acid strand, wherein the detector strand comprises a functional group for conjugating with an electroactive label; (b) contacting the CRISPR detection system or detector strand from (a) with an electrode, wherein the electrode comprises: (i) a conductive surface, (ii) a nanocomposite coating comprising a mixture of a conducting element and a denatured proteinaceous material coated on at least a part of said conductive surface, and (iii) an effector nucleic acid strand immobilized on the conductive surface, wherein the effector strand is substantially complementary to the detector strand; (c) conjugating the detector nucleic acid strand with at least one electroactive label; and (d) applying a voltage to the electrode and measuring a current generated from electrode.

Embodiment 57: A method for detecting a nucleic acid, the method comprising: (a) contacting a target nucleic acid with a detection CRISPR system, wherein the CRISPR detection system comprises an effector protein, one or more guide nucleic acid strands designed to bind to the target nucleic acid molecules, and a detector nucleic acid strand, wherein the detector strand is conjugated with at least one electroactive label; (b) contacting the sample with the CRISPR detection system or detector strand with an electrode, wherein the electrode comprises: (i) a conductive surface, (ii) a nanocomposite coating comprising a mixture of a conducting element and a denatured proteinaceous material coated on at least a part of said conductive surface, and (iii) an effector nucleic acid strand immobilized on the conductive surface, wherein the effector strand is substantially complementary to the detector strand; and (c) applying a voltage to the electrode and measuring a current generated from electrode.

Embodiment 58: A method for detecting a nucleic acid, the method comprising: (a) contacting a target nucleic acid with a detection CRISPR system, wherein the CRISPR detection system comprises an effector protein and one or more guide nucleic acid strands designed to bind to or hybridized with the target nucleic acid molecules; (b) contacting the CRISPR detection system from (a) with an electrode, wherein the electrode comprises: (i) a conductive surface, (ii) a nanocomposite coating comprising an allotrope of carbon having atoms arranged in a hexagonal lattice and a denatured proteinaceous material coated on at least a part of said conductive surface, and (iii) an effector nucleic acid strand immobilized on the conductive surface, wherein the effector strand is substantially complementary to the detector strand and comprises an electroactive label; and (c) applying a voltage to the electrode and measuring a current generated from electrode.

Embodiment 59: A method for detecting a nucleic acid, the method comprising: (a) contacting a target nucleic acid with a detection CRISPR system, wherein the CRISPR detection system comprises an effector protein and one or more guide nucleic acid strands designed to bind to or hybridized with the target nucleic acid molecules; (b) contacting the CRISPR detection system from (a) with an electrode, wherein the electrode comprises: (i) a conductive surface; (ii) a nanocomposite coating comprising an allotrope of carbon having atoms arranged in a hexagonal lattice and a denatured proteinaceous material coated on at least a part of said conductive surface; and (iii) an effector nucleic acid strand immobilized on the conductive surface, wherein the effector strand is substantially complementary to the detector strand and comprises a functional group for conjugating with an electroactive label; (c) conjugating the effector strand with an electroactive label; and (d) applying a voltage to the electrode and measuring a current generated from electrode.

Embodiment 60: A method for detecting a nucleic acid, the method comprising: (a) contacting a target nucleic acid with a detection CRISPR system, wherein the CRISPR detection system comprises an effector protein, one or more guide nucleic acid strands designed to bind to or hybridized with the target nucleic acid molecules, and an effector strand comprises an electroactive label and a functional group for immobilizing the effector strand on an electrode; (b) immobilizing the effector strand from (a) on an electrode, wherein the electrode comprises: (i) a conductive surface; and (ii) a nanocomposite coating comprising an allotrope of carbon having atoms arranged in a hexagonal lattice and a denatured proteinaceous material coated on at least a part of said conductive surface; and (c) applying a voltage to the electrode and measuring a current generated from electrode.

Embodiment 61: A method for detecting a nucleic acid, the method comprising: (a) contacting a target nucleic acid with a detection CRISPR system, wherein the CRISPR detection system comprises an effector protein, one or more guide nucleic acid strands designed to bind to or hybridized with the target nucleic acid molecules, and an effector strand comprises a functional group for immobilizing the effector strand on an electrode and a functional group for conjugating with an electroactive label; (b) immobilizing the effector strand from (a) on an electrode, wherein the electrode comprises: (i) a conductive surface; and (ii) a nanocomposite coating comprising an allotrope of carbon having atoms arranged in a hexagonal lattice and a denatured proteinaceous material coated on at least a part of said conductive surface; (c) conjugating the effector strand immobilized on the electrode with an electroactive label; and (d) applying a voltage to the electrode and measuring a current generated from electrode.

Embodiment 62: The method of any one of Embodiments 55-61, wherein the method comprises contacting an electroactive mediator precipitating composition with the electrode prior to or simultaneously with the applying the voltage step.

Embodiment 63: The method of any one of Embodiments 55-62, wherein the method further comprises amplifying the target nucleic acid prior to contacting with the CRISPR detection system.

Embodiment 64: The method of Embodiment 63, wherein said amplification comprises isothermal amplification.

Embodiment 65: The method of Embodiment 63 or 64, wherein the method further comprises a reverse-transcriptase step prior to amplification.

Embodiment 66: The method of any one of Embodiments 55-65, wherein the method further comprises a step of extracting the target nucleic acid from a sample.

Embodiment 67: The method of any one of Embodiments 55-66, wherein the method further comprises one or more steps of washing the electrode.

Embodiment 68: The method of any one of Embodiments 55-67, wherein the nanocomposite coating comprises a three dimensional, porous matrix.

Embodiment 69: A kit comprising the nucleic acid detection system of any one of Embodiments 1-54.

Embodiment 70: A composition comprising a nucleic acid detection system of any one of Embodiments 1-54.

Embodiment 71: Use of a nucleic acid system of any one of Embodiments 1-54 for detecting a target nucleic acid.

Embodiment 72: Use of a nucleic acid system of any one of Embodiments 1-54 for detecting a target nucleic acid according to a method of any one of Embodiments 55-68.

Some additional exemplary embodiments can be described as follows:

Embodiment A: A nucleic acid detection system comprising: (a) a detection CRISPR system comprising an effector protein and one or more guide nucleic acid strands designed to bind to corresponding target nucleic acid molecules; (b) an electrode comprising: (i) a conductive surface; (ii) a nanocomposite coating comprising an allotrope of carbon having atoms arranged in a hexagonal lattice and a denatured proteinaceous material coated on at least a part of said conductive surface; and (iii) an effector nucleic acid strand on the conductive surface; and (c) a detector nucleic acid strand, wherein the detector nucleic acid stand is substantially complementary to the effector nucleic acid strand and is optionally conjugated with at least electroactive label.

Embodiment B: The nucleic acid detection system of Embodiment A, wherein the CRISPR system effector protein is selected from the group consisting of Cas1, Cas1B, Cas2, Cas3, Cas4, Cas5, Cash, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and homologues thereof, or modified versions thereof.

Embodiment C: The nucleic acid detection system of any one of Embodiments A-B, wherein the CRISPR system effector protein is selected from the group consisting of Cas 9, Cas12a, Cas13a, and Cas14.

Embodiment D: The nucleic acid detection system of any one of Embodiments A-C, wherein the CRISPR system effector protein is a DNA targeting protein.

Embodiment E: The nucleic acid detection system of any one of Embodiments A-D, wherein the electroactive label is an enzyme or a metallocene.

Embodiment F: The nucleic acid detection system of any one of Embodiments A-E, wherein the electrode is comprised in an electrochemical sensor.

Embodiment G: The nucleic acid detection system of any one of Embodiments A-F further comprising an electroactive mediator precipitating composition.

Embodiment H: The nucleic acid detection system of any one of Embodiments A-G, wherein the nanocomposite coating comprise carbon nanotubes, graphene and/or reduced graphene oxide mixed with denatured Bovine Serum Albumin (BSA).

Embodiment I: A method for detecting a nucleic acid, the method comprising: (a) incubating a sample suspected of comprising a target nucleic acid with a detection CRISPR system, wherein the CRISPR detection system comprises an effector protein and one or more guide nucleic acid strands designed to bind to the target nucleic acid molecules; (b) contacting the sample with the CRISPR detection system with an electrode, wherein the electrode comprises: (i) a conductive surface, (ii) a nanocomposite coating comprising an allotrope of carbon having atoms arranged in a hexagonal lattice and a denatured proteinaceous material coated on at least a part of said conductive surface, and (iii) an effector nucleic acid strand on the conductive surface; (c) contacting the electrode with a detector nucleic acid strand, wherein the detector nucleic acid stand is substantially complementary to the effector nucleic acid strand and is conjugated with at least one electroactive label; and (d) applying a voltage to the electrode and measuring a current generated from electrode.

Embodiment J: The method of Embodiment I, wherein the method comprises contacting an electroactive mediator precipitating composition with the electrode prior to step (d).

Embodiment K: The method of any one of Embodiments I-J, wherein the method further comprises amplifying the target nucleic acid prior to incubating with the CRISPR detection system target nucleic acid.

Embodiment L: The method of Embodiment K, wherein said amplification comprises isothermal amplification.

Embodiment M: The method of Embodiment K or L, wherein the method further comprises a reverse-transcriptase step prior to amplification.

Embodiment N: The method of any one of Embodiments I-M, wherein the method further comprises a step of extracting the target nucleic acid from a sample.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Some Selected Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages can mean±5% (e.g., ±4%, ±3%, ±2%, ±1%) of the value being referred to.

Where a range of values is provided, each numerical value between the upper and lower limits of the range is contemplated and disclosed herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples in no way should be construed as being further limiting.

### Example

CRISPR-based diagnostic readouts have been typically limited to fluorescence. This work describes a novel and nonobvious approach to obtain electrochemical readouts from CRISPR/Cas-based diagnostics.

### Materials and Methods

Preparation of the chip: Gold chips were prepared using standard photolithography process by depositing 20 nm of titanium and 150 nm of gold on a glass wafer, as described previously (Sabate Del Rio, J.; Henry, O. Y. F.; Jolly, P.; Ingber, D. E., An antifouling coating that enables affinity-based electrochemical biosensing in complex biological fluids. Nat Nanotechnol 2019, 14 (12), 1143-1149)¹. Prior to use, gold chips were cleaned by 5 min sonication in acetone and then in isopropanol. To ensure a clean surface, the chips were then treated with oxygen plasma using a Zepto Diener plasma cleaner (Diener Electronics, Germany) at 0.5 mbar and 50% power for 2 min.

Nanocomposite preparation: Amine-functional reduced graphene oxide (Sigma Aldrich, USA) was mixed with 5 mgmL⁻¹ BSA (Sigma Aldrich, USA) in PBS solution, ultrasonicated for 1 h using 1 s on/off cycles, heated at 105° C. for 5 min to denature the protein and centrifuged to remove the excess aggregates. The nanomaterial solution was then mixed with 70% glutaraldehyde (Sigma Aldrich, USA) for crosslinking in the ratio of 69:1, deposited on the glass chip with gold electrodes and incubated in the humidity chamber for 20-24 h to form a conductive nanocomposite (WO2019023567)².

### SHERLOCK Lyme Disease Assays (Fluorescent Readout Controls):

Inventors designed five gRNAs (sensors) to target four different Lyme-associated genes: ospC (sensor a), BB0631 (sensor b), BB0476—tuf (sensor c), and two different regions of BB0477—rpsJ (sensors d and e). Synthetic representations (triggers) of the targeted DNAs were generated via PCR, and the gRNAs were produced by in vitro transcription. Each gRNA was incubated with Cas12a for 10 min at 37° C. prior to the addition of the quenched ssDNA reporter and trigger DNA. All five selected gRNA sensors were able to successfully detect their corresponding DNA triggers as reflected by the fluorescent signal increase caused by cleavage of the reporter ssDNA. Some exemplary sequences used in this study are shown in Table 1.

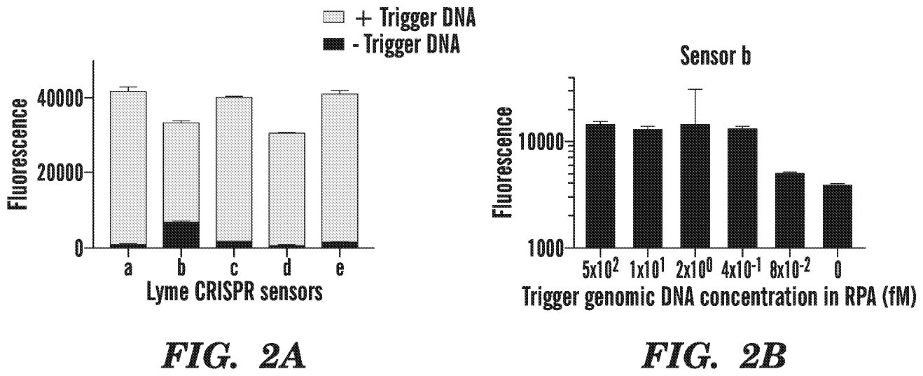

Results are shown in FIG. 2A. As can be seen, the Cas12a/gRNA-based system was able to detect low picomolar concentrations of target dsDNA.

In order to assess capacity to detect Borrelia spirochetes, inventors tested the performance of the assays using full-length genomic DNA (gDNA) purified from the bacteria. Serial dilutions of genomic DNA were prepared, subjected the samples to RPA, and applied the SHERLOCK detection system. As can be seen from FIG. 2B, double-digit attomolar concentrations of gDNA could be detected using sensor b.

TABLE 1
Sequences used in this study
Newly developed gRNA sequences, target sequences and RPA amplification
primers for detection of Lyme disease by using a Cas12a enzyme.

| gRNA | | | | |
|---|---|---|---|---|
| name: | gRNA sequence: | Target sequence: | RPA-Forward | RPA-reverse |
| b | GTAATTTCTACTA | TGGGCAGATCTC | TGCAGCGAT | ACCAACACAAT |
| | AGTGTAGATGAGT | TTCTACTCCAAA | TTCTGTACTT | AGCAAAAGAA |
| | AGAAGAGATCTGC | (SEQ ID NO: 2) | TGTTCATCG | AATGTAATTC |
| | CCA (SEQ ID NO: 1) | | CAAATAC | (SEQ ID NO: 4) |
| | | | (SEQ ID NO: 3) | |

### SHERLOCK Lyme Disease Assays (Electrochemistry Readout)

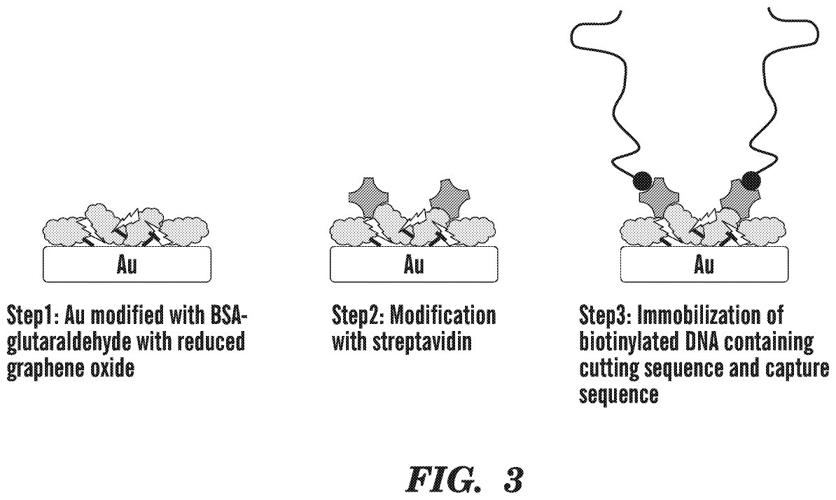

Functionalization of Chip: Nanocomposite coated chips were functionalized with streptavidin through EDC/NHS activation. The chips were incubated with 400 mM of EDC and 200 mM of NHS in 0.1 M MES buffer pH 6 for 30 minutes, rinsed with ultrapure water and dried. A solution of 100 μg·mL⁻¹ streptavidin in MES was deposited on specific working electrodes. The chips were later incubated in a water saturated atmosphere overnight at 4° C., rinsed and washed with PBS buffer in a shaker for 30 minutes. Then, 10 μL of a 1 M ethanolamine in PBS adjusted to pH 7.4 with HCl was drop-casted on each electrode and incubated at room temperature for 30 minutes. Chips were thoroughly rinsed and exposed to 0.1 μM biotinylated ssDNA in PBS for 1 hour (FIG. 3). Electrodes were used after the final biotin-ssDNA conjugation step.

The two reporter ssDNA sequences were partially complementary (Table 2), one sequence had a thiol modification (to attach to HRP), while the other sequence contained a biotin modification (for streptavidin conjugation). The thiolated ssDNA sequence was conjugated to HRP using EZ-Link™ Maleimide Activated Horseradish Peroxidase (HRP) from Thermo Fisher. 100 uM DNA solution was mixed the activated HRP and was left overnight to react at room temperature. Thereafter, the unreacted DNA was removed using a spin column.

TABLE 2
Electrochemistry sequences used for Lyme

| | Biotinylated sequence | Thiolated sequence |
|---|---|---|
| Lyme disease | 5Biosg/TT TTT TTT TTT TTT TAA | /5ThioMC6-D/TT TTT TTT TAG |
| electrochemistry | TAA TAA TAA TAA TAA TAA | AAG AAG AAG AAG A (SEQ ID |
| assay | TCT TCT TCT TCT TCT (SEQ ID | NO: 6) |
| | NO: 5) | |

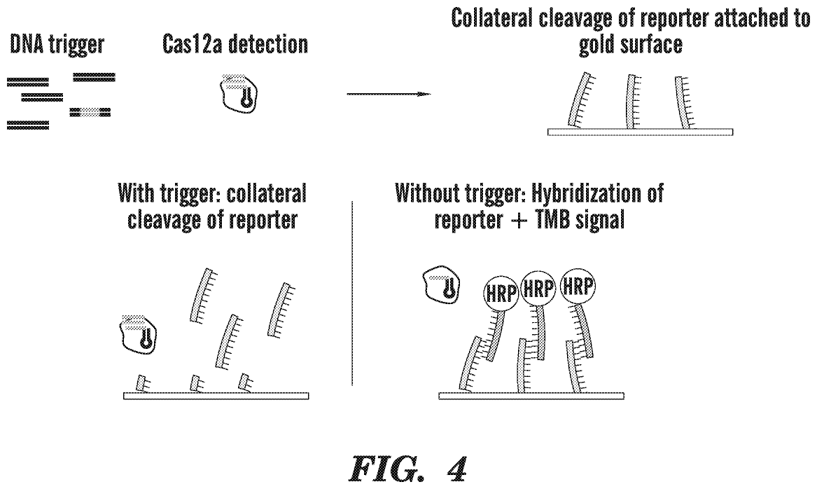

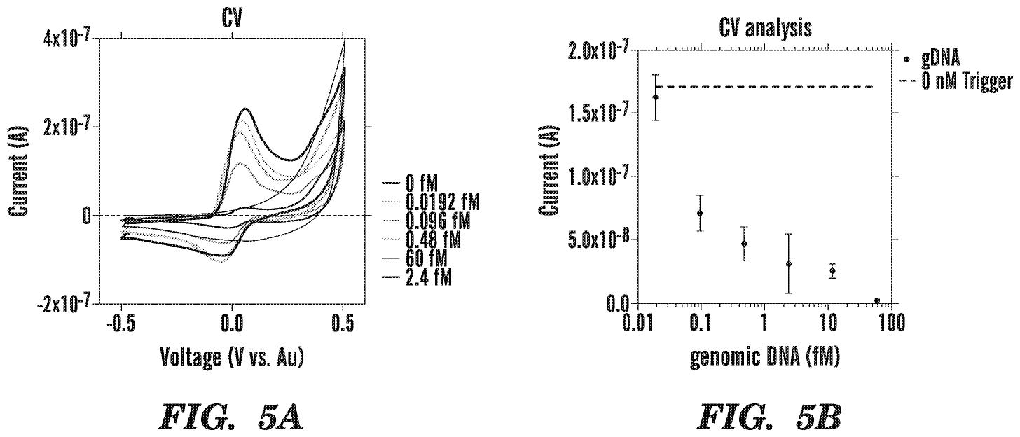

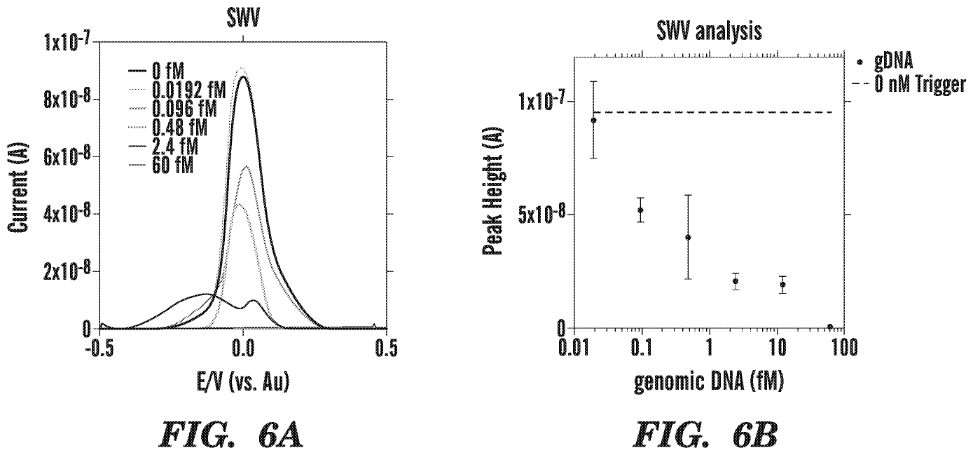

Lyme CRISPR-electrochemistry assays: Genomic DNA was serially diluted and amplified with RPA liquid kit (Twist Dx) following the kit instructions at 37 C. Briefly, 1 μl of the diluted genomic DNA was added to 19 μl of RPA liquid basic (TwistDx) reactions that contained 480 μM of each RPA primer b (Table 1) and 14 mM magnesium acetate, as per manufacturer's instructions. The RPA reaction was incubated for 40 min at 37° C. After amplification, the gRNA-b and Cas12a were added to final concentrations of 25 nM and 30 nM, respectively. RPA/Cas mixture was deposited on the electrodes and incubated for 1 h, during which time the biotinylated sequence was cleaved when the trigger sequence was present in the mixture (Cas enzyme was activated). After that, chips were rinsed and incubated with the HRP-conjugated reporter sequence for 30 min (FIG. 4). Thereafter, the chips were washed and incubated with TMB for 1 minute. Final measurement was then performed in PBST using a potentiostat (Autolab PGSTAT128N, Metrohm; VSP, Bio-Logic) by a CV scan with 1 V/s scan rate between −0.5 and 0.5 V vs on-chip integrated gold quasi reference electrode. Peak area was calculated using Nova 1.11 software. Both cyclic voltammetry (FIGS. 5A and 4B) and square wave voltammetry (FIGS. 6A and 6B) allowed the measurement of attomolar concentrations of Lyme-specific target dsDNA. To date, there are no reports of such a sensitive Cas-mediated detection system with an electrical readout.

### COVID-19 Diagnostic

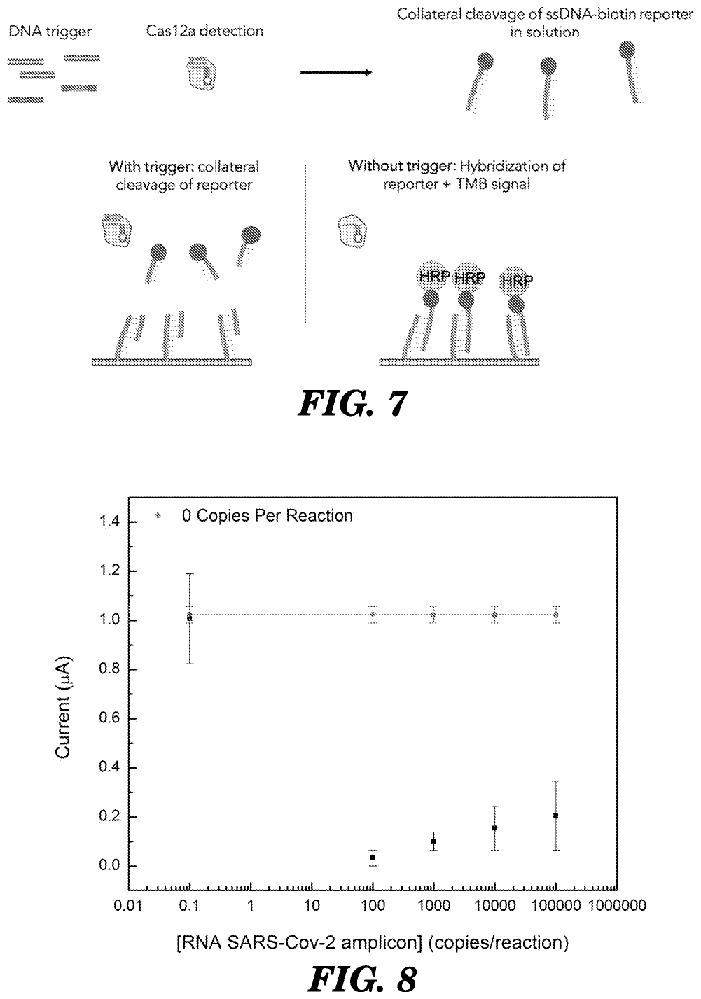

For a second study, amine terminated peptide nucleic acid sequence (AEEA-ACAACAACAACAACA (SEQ ID NO: 7)) where AEEA is an O-linker and is used as a spacer, was conjugated to the electrode, and the other was a ssDNA (sequence/5Biosg/AT TAT TAT TAT TAT TTG TTG TTG TTG TTG T (SEQ ID NO: 8)) conjugated to a biotin that bound to poly-streptavidin-HRP. Upon Cas12a activation, the ssDNA-biotin reporter is cleaved in solution, thus preventing binding to the complementary PNA sequence on the surface. Poly-streptavidin-HRP is then added and able to bind to the ssDNA reporter-biotin. The concentration of HRP bound to the electrode was read by HRP-dependent oxidation of TMB (TMB enhanced one component, Sigma). TMB enhanced forms an insulating, non-soluble layer on the electrode surface (FIG. 7).

CoV-2 CRISPR-electrochemistry assays: Genomic RNA was serially diluted and amplified with 2×LAMP master mix (NEB) for 40 min at 65 C. Briefly, 8 W of the diluted genomic DNA was added to 2 μl of the 10× primer mix (table 4) and 10 μl of the LAMP master mix (NEB). LAMP mixtures were incubated for 40 min at 65 C. After LAMP amplification, 4 μl of the amplified LAMP mixture were mixed with 10 μl of nuclease-free water and 5 ul of Cas mix: 20 nM reporter, 50 nM Cas, 62.5 nM gRNA (table 3) in 10×NEB 2.1 buffer. Mixtures were incubated for 1 h at 37 C during which time the ssDNA biotinylated reporter (sequence/5Biosg/AT TAT TAT TAT TAT TTG TTG TTG TTG TTG T SEQ ID NO: 8)) was cleaved. After that, 15 uL of the LAMP/reporter/Cas mixtures were deposited on the chips for 30 min. Thereafter, the chips were washed and incubated with TMB for 1 minute. Final measurement was then performed in PBST using a potentiostat (Autolab PGSTAT128N, Metrohm; VSP, Bio-Logic) by a CV scan with 1 V/s scan rate between −0.5 and 0.5 V vs on-chip integrated gold quasi reference electrode. Peak area was calculated using Nova 1.11 software. Cyclic voltammetry (FIG. 8) allowed the measurement of attomolar concentrations of SARS-CoV-2 target RNA.

TABLE 3
Cas12a gRNA and target regions for SARS-CoV-2 N2 gene.

| | gRNA-4 | Target |
|---|---|---|
| CoV-2 | GTAATTTCTACTAAGTGTAGATC | TTTGCCCCCAGCGCTTCAGCGTTC |
| assay | CCCCAGCGCTTCAGCGTTC (SEQ ID | (SEQ ID NO: 10) |
| | NO: 9) | |

TABLE 4
LAMP primer sequences and concentrations in LAMP assays.

| | 10X | 1X | |
|---|---|---|---|
| | CONCENTRATION | CONCENTRATION | |
| PRIMER | (STOCK) | (FINAL) | sequence |
| FIP | 16 μM | 1.6 μM | GCG GCC AAT GTT TGT |
| | | | AAT CAG TAG ACG TGG |
| | | | TCC AGA ACA A (SEQ ID |
| | | | NO: 11) |
| BIP | 16 μM | 1.6 μM | TCA GCG TTC TTC GGA |
| | | | ATG TCG CTG TGT AGG |
| | | | TCA ACC ACG (SEQ ID NO: |
| | | | 12) |
| F3 | 2 μM | 0.2 μM | GCT GCT GAG GCT TCT |
| | | | AAG (SEQ ID NO: 13) |
| B3 | 2 μM | 0.2 μM | GCG TCA ATA TGC TTA |
| | | | TTC AGC (SEQ ID NO: 14) |
| LOOP F | 4 μM | 0.4 μM | CCT TGT CTG ATT AGT |
| | | | TCC TGG T (SEQ ID NO: 15) |
| LOOP B | 4 μM | 0.4 μM | TGG CAT GGA AGT CAC |
| | | | ACC (SEQ ID NO: 16) |

All patents, patent applications, and publications identified are expressly incorporated herein by reference for the purpose of describing and disclosing, for example, the methodologies described in such publications that can be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A nucleic acid detection system comprising:
a. a detection CRISPR system comprising an effector protein and one or more guide nucleic acid strands designed to bind to corresponding target nucleic acid molecules;
b. an effector nucleic acid strand;
c. a detector nucleic acid strand substantially complementary to the effector nucleic acid strand and conjugated with at least one electroactive label; and
d. an electrode comprising:
i. a conductive surface; and
ii. a nanocomposite coating comprising a mixture of a conducting element and a denatured proteinaceous material coated on at least a part of said conductive surface.

2. (canceled)

3. The nucleic acid detection system of claim 2, wherein the CRISPR system effector protein is selected from the group consisting of Cas 9, Cas12a, Cas13a, and Cas14.

4. (canceled)

5. The nucleic acid detection system of claim 1, wherein the CRISPR system effector protein is a DNA targeting protein or an RNA targeting protein.

6. (canceled)

7. The nucleic acid detection system of claim 1, wherein the effector strand further comprises:
a. a functional group for immobilization on the conductive surface or for conjugating with at least one electroactive label;
b. at least one electroactive label; or
c. a nucleic acid modification.

8. The nucleic acid detection system of claim 7, wherein the functional group is at the 5′-end of the effector strand or the 3′-end of the effector strand.

9.-15. (canceled)

16. The nucleic acid detection system of claim 7, wherein the nucleic acid modification inhibits or reduces cleavage of the effector strand by the CRISPR system effector protein.

17. The nucleic acid detection system of claim 16, wherein the nucleic acid modification that inhibits or reduces cleavage of the effector strand by the CRISPR system effector protein is a peptide nucleic acid (PNA).

18. The nucleic acid detection system of claim 1, wherein the detector strand further comprises:
a. a functional group for conjugating with the at least one electroactive label;
b. at least one electroactive label; or
c. a nucleic acid modification.

19. The nucleic acid detection system of claim 18, wherein the functional group for conjugating with the electroactive label is at the 5′-end of the detector strand or at the 3′-end of the detector strand.

20.-22. (canceled)

23. The nucleic acid detection system of claim 18, wherein the nucleic acid modification inhibits or reduces cleavage of the detector strand by the CRISPR system effector protein.

24. The nucleic acid detection system of claim 23, wherein the nucleic acid modification that inhibits or reduces cleavage of the detector strand by the CRISPR system effector protein is a peptide nucleic acid.

25. The nucleic acid detection system of claim 1, wherein the electrode is comprised in an electrochemical cell.

26.-27. (canceled)

28. The nucleic acid detection system of claim 1, wherein the conducting element comprises:
a. conductive and semi-conductive particles, rods, fibers, nano-particles or polymers;
b. gold; or
c. an allotrope of carbon atoms arranged in a hexagonal lattice.

29.-30. (canceled)

31. The nucleic acid detection system of claim 28, wherein the allotrope of carbon is:
a. a functionalized material; or
b. carbon nanotubes comprising carboxylated carbon nanotubes (CNTs) or aminated carbon nanotubes, reduced graphene oxide comprising carboxylated reduced graphene oxide or an aminated reduced graphene oxide, or mixtures thereof.

32.-34. (canceled)

35. The nucleic acid detection system of claim 1, wherein the electroactive label comprises:
a. an enzyme selected from horseradish peroxidase (HRP), alkaline phosphatase (AP), glucose oxidase (GOx), tyrosinase, urease, a DNAzyme, an aptazyme, or any combinations thereof; or
b. or a metallocene selected from ferrocene, cobaltocene, nickelocene, ruthenocene, vanadocene, chromocene, decamethylmanganocene, decamethylrhenocene, tungstencense, titanocene, or zirconocene.

36.-40. (canceled)

41. The nucleic acid detection system of claim 1, wherein the system further comprises an electroactive mediator precipitating composition comprising:
a. a reporter enzyme substrate selected from the group consisting of hydrogen peroxide, carbamide peroxide, nucleotides, oligonucleotides, RNA, DNA, phosphorylated peptides, phosphorylated proteins, phosphorylated small molecules, glucose, phenols, tyrosine, dopamine, catechol, urea, and any combination thereof;
b. an electroactive mediator selected from the group consisting of 3,3′,5,5′-tetramethylbenzidine (TMB), o-phenylenedi amine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof; and/or
c. a precipitating agent selected from the group consisting of a water-soluble polymer, a pyrrolidinone polymer, a polyaniline, a polypyrrole, a polythiophene, alginic acid, methyl vinyl ether/maleic anhydride copolymer, dextran sulfate, carrageenan, and any combination thereof.

42.-52. (canceled)

53. The nucleic acid detection system of claim 1, wherein the system further comprises a target nucleic acid.

54. The nucleic acid detection system of claim 1, wherein the nanocomposite coating comprises a three dimensional, porous matrix.

55. A method for detecting a nucleic acid, the method comprising:
a. contacting a target nucleic acid with a detection CRISPR system, wherein the CRISPR detection system comprises an effector protein and one or more guide nucleic acid strands designed to bind to or hybridize with the target nucleic acid molecules;
b. contacting the CRISPR detection system from (a) with an electrode, wherein the electrode comprises:
i. a conductive surface;
ii. a nanocomposite coating comprising a mixture of a conducting element and a denatured proteinaceous material coated on at least a part of said conductive surface; and
iii. an effector nucleic acid strand immobilized on the conductive surface;
c. contacting the electrode with a detector nucleic acid strand, wherein the detector nucleic acid stand is substantially complementary to the effector nucleic acid strand and is conjugated with at least one electroactive label; and
d. applying a voltage to the electrode and measuring a current generated from electrode.

56. (canceled)

57. A method for detecting a nucleic acid, the method comprising:
a. contacting a target nucleic acid with a detection CRISPR system, wherein the CRISPR detection system comprises an effector protein, one or more guide nucleic acid strands designed to bind to the target nucleic acid molecules, and a detector nucleic acid strand, wherein the detector strand is conjugated with at least one electroactive label;
b. contacting the detector strand from (a) with an electrode, wherein the electrode comprises:
i. a conductive surface;
ii. a nanocomposite coating comprising a mixture of a conducting element and a denatured proteinaceous material coated on at least a part of said conductive surface; and
iii. an effector nucleic acid strand immobilized on the conductive surface, wherein the effector strand is substantially complementary to the detector strand; and
c. applying a voltage to the electrode and measuring a current generated from electrode.

58.-72. (canceled)


SEQUENCE LISTING (16 sequences)

SEQ ID NO: 1 - DNA, 42 bp, Artificial Sequence
  synthetic oligonucleotide
gtaatttctactaagtgtagatgagtagaagagatctgccca

SEQ ID NO: 2 - DNA, 24 bp, Artificial Sequence
  synthetic oligonucleotide
tgggcagatctcttctactccaaa

SEQ ID NO: 3 - DNA, 34 bp, Artificial Sequence
  synthetic oligonucleotide
gcagcgatttctgtactttgttcatcgcaaatac

SEQ ID NO: 4 - DNA, 31 bp, Artificial Sequence
  synthetic oligonucleotide
accaacacaatagcaaaagaaaatgtaattc

SEQ ID NO: 5 - DNA, 50 bp, Artificial Sequence
  synthetic oligonucleotide
tttttttttttttttaataataataataataataatcttcttcttcttct

SEQ ID NO: 6 - DNA, 24 bp, Artificial Sequence
  synthetic oligonucleotide
tttttttttagaagaagaagaaga

SEQ ID NO: 7 - DNA, 15 bp, Artificial Sequence
  synthetic oligonucleotide
acaacaacaacaaca

SEQ ID NO: 8 - DNA, 30 bp, Artificial Sequence
  synthetic oligonucleotide
attattattattatttgttgttgttgttgt

SEQ ID NO: 9 - DNA, 42 bp, Artificial Sequence
  synthetic oligonucleotide
gtaatttctactaagtgtagatcccccagcgcttcagcgttc

SEQ ID NO: 10 - DNA, 24 bp, Artificial Sequence
  synthetic oligonucleotide
tttgcccccagcgcttcagcgttc

SEQ ID NO: 11 - DNA, 40 bp, Artificial Sequence
  synthetic oligonucleotide
gcggccaatgtttgtaatcagtagacgtggtccagaacaa

SEQ ID NO: 12 - DNA, 39 bp, Artificial Sequence
  synthetic oligonucleotide
tcagcgttcttcggaatgtcgctgtgtaggtcaaccacg

SEQ ID NO: 13 - DNA, 18 bp, Artificial Sequence
  synthetic oligonucleotide
gctgctgaggcttctaag

SEQ ID NO: 14 - DNA, 21 bp, Artificial Sequence
  synthetic oligonucleotide
gcgtcaatatgcttattcagc

SEQ ID NO: 15 - DNA, 22 bp, Artificial Sequence
  synthetic oligonucleotide
ccttgtctgattagttcctggt

SEQ ID NO: 16 - DNA, 18 bp, Artificial Sequence
  synthetic oligonucleotide
tggcatggaagtcacacc